SEQUENCE LISTING (5 sequences)

SEQ ID NO: 1 - DNA, 2412 bp, Homo sapiens
atgagggccctgtgggtgctgggcctctgctgcgtcctgctgaccttcgggtcggtcagagctgacgatgaagttgatgtggatggtacagtagaagaggatctgggtaaaagtagagaaggatcaaggacggatgatgaagtagtacagagagaggaagaagctattcagttggatggattaaatgcatcacaaataagagaacttagagagaagtcggaaaagtttgccttccaagccgaagttaacagaatgatgaaacttatcatcaattcattgtataaaaataaagagattttcctgagagaactgatttcaaatgcttctgatgctttagataagataaggctaatatcactgactgatgaaaatgctctttctggaaatgaggaactaacagtcaaaattaagtgtgataaggagaagaacctgctgcatgtcacagacaccggtgtaggaatgaccagagaagagttggttaaaaaccttggtaccatagccaaatctgggacaagcgagtttttaaacaaaatgactgaagcacaggaagatggccagtcaacttctgaattgattggccagtttggtgtcggtttctattccgccttccttgtagcagataaggttattgtcacttcaaaacacaacaacgatacccagcacatctgggagtctgactccaatgaattttctgtaattgctgacccaagaggaaacactctaggacggggaacgacaattacccttgtcttaaaagaagaagcatctgattaccttgaattggatacaattaaaaatctcgtcaaaaaatattcacagttcataaactttcctatttatgtatggagcagcaagactgaaactgttgaggagcccatggaggaagaagaagcagccaaagaagagaaagaagaatctgatgatgaagctgcagtagaggaagaagaagaagaaaagaaaccaaagactaaaaaagttgaaaaaactgtctgggactgggaacttatgaatgatatcaaaccaatatggcagagaccatcaaaagaagtagaagaagatgaatacaaagctttctacaaatcattttcaaaggaaagtgatgaccccatggcttatattcactttactgctgaaggggaagttaccttcaaatcaattttatttgtacccacatctgctccacgtggtctgtttgacgaatatggatctaaaaagagcgattacattaagctctatgtgcgccgtgtattcatcacagacgacttccatgatatgatgcctaaatacctcaattttgtcaagggtgtggtggactcagatgatctccccttgaatgtttcccgcgagactcttcagcaacataaactgcttaaggtgattaggaagaagcttgttcgtaaaacgctggacatgatcaagaagattgctgatgataaatacaatgatactttttggaaagaatttggtaccaacatcaagcttggtgtgattgaagaccactcgaatcgaacacgtcttgctaaacttcttaggttccagtcttctcatcatccaactgacattactagcctagaccagtatgtggaaagaatgaaggaaaaacaagacaaaatctacttcatggctgggtccagcagaaaagaggctgaatcttctccatttgttgagcgacttctgaaaaagggctatgaagttatttacctcacagaacctgtggatgaatactgtattcaggcccttcccgaatttgatgggaagaggttccagaatgttgccaaggaaggagtgaagttcgatgaaagtgagaaaactaaggagagtcgtgaagcagttgagaaagaatttgagcctctgctgaattggatgaaagataaagcccttaaggacaagattgaaaaggctgtggtgtctcagcgcctgacagaatctccgtgtgctttggtggccagccagtacggatggtctggcaacatggagagaatcatgaaagcacaagcgtaccaaacgggcaaggacatctctacaaattactatgcgagtcagaagaaaacatttgaaattaatcccagacacccgctgatcagagacatgcttcgacgaattaaggaagatgaagatgataaaacagttttggatcttgctgtggttttgtttgaaacagcaacgcttcggtcagggtatcttttaccagacactaaagcatatggagatagaatagaaagaatgcttcgcctcagtttgaacattgaccctgatgcaaaggtggaagaagagcccgaagaagaacctgaagagacagcagaagacacaacagaagacacagagcaagacgaagatgaagaaatggatgtgggaacagatgaagaagaagaaacagcaaaggaatctacagctgaaaaagatgaattgtaa

SEQ ID NO: 2 - protein, 803 aa, Homo sapiens
MRALWVLGLCCVLLTFGSVRADDEVDVDGTVEEDLGKSREGSRTDDEVVQREEEAIQLDGLNASQIRELREKSEKFAFQAEVNRMMKLIINSLYKNKEIFLRELISNASDALDKIRLISLTDENALSGNEELTVKIKCDKEKNLLHVTDTGVGMTREELVKNLGTIAKSGTSEFLNKMTEAQEDGQSTSELIGQFGVGFYSAFLVADKVIVTSKHNNDTQHIWESDSNEFSVIADPRGNTLGRGTTITLVLKEEASDYLELDTIKNLVKKYSQFINFPIYVWSSKTETVEEPMEEEEAAKEEKEESDDEAAVEEEEEEKKPKTKKVEKTVWDWELMNDIKPIWQRPSKEVEEDEYKAFYKSFSKESDDPMAYIHFTAEGEVTFKSILFVPTSAPRGLFDEYGSKKSDYIKLYVRRVFITDDFHDMMPKYLNFVKGVVDSDDLPLNVSRETLQQHKLLKVIRKKLVRKTLDMIKKIADDKYNDTFWKEFGTNIKLGVIEDHSNRTRLAKLLRFQSSHHPTDITSLDQYVERMKEKQDKIYFMAGSSRKEAESSPFVERLLKKGYEVIYLTEPVDEYCIQALPEFDGKRFQNVAKEGVKFDESEKTKESREAVEKEFEPLLNWMKDKALKDKIEKAVVSQRLTESPCALVASQYGWSGNMERIMKAQAYQTGKDISTNYYASQKKTFEINPRHPLIRDMLRRIKEDEDDKTVLDLAVVLFETATLRSGYLLPDTKAYGDRIERMLRLSLNIDPDAKVEEEPEEEPEETAEDTTEDTEQDEDEEMDVGTDEEEETAKESTAEKDEL

SEQ ID NO: 3 - protein, 4 aa, Homo sapiens
KDEL

SEQ ID NO: 4 - protein, 799 aa, Homo sapiens
MRALWVLGLCCVLLTFGSVRADDEVDVDGTVEEDLGKSREGSRTDDEVVQREEEAIQLDGLNASQIRELREKSEKFAFQAEVNRMMKLIINSLYKNKEIFLRELISNASDALDKIRLISLTDENALSGNEELTVKIKCDKEKNLLHVTDTGVGMTREELVKNLGTIAKSGTSEFLNKMTEAQEDGQSTSELIGQFGVGFYSAFLVADKVIVTSKHNNDTQHIWESDSNEFSVIADPRGNTLGRGTTITLVLKEEASDYLELDTIKNLVKKYSQFINFPIYVWSSKTETVEEPMEEEEAAKEEKEESDDEAAVEEEEEEKKPKTKKVEKTVWDWELMNDIKPIWQRPSKEVEEDEYKAFYKSFSKESDDPMAYIHFTAEGEVTFKSILFVPTSAPRGLFDEYGSKKSDYIKLYVRRVFITDDFHDMMPKYLNFVKGVVDSDDLPLNVSRETLQQHKLLKVIRKKLVRKTLDMIKKIADDKYNDTFWKEFGTNIKLGVIEDHSNRTRLAKLLRFQSSHHPTDITSLDQYVERMKEKQDKIYFMAGSSRKEAESSPFVERLLKKGYEVIYLTEPVDEYCIQALPEFDGKRFQNVAKEGVKFDESEKTKESREAVEKEFEPLLNWMKDKALKDKIEKAVVSQRLTESPCALVASQYGWSGNMERIMKAQAYQTGKDISTNYYASQKKTFEINPRHPLIRDMLRRIKEDEDDKTVLDLAVVLFETATLRSGYLLPDTKAYGDRIERMLRLSLNIDPDAKVEEEPEEEPEETAEDTTEDTEQDEDEEMDVGTDEEEETAKESTAE

SEQ ID NO: 5 - protein, 217 aa, Homo sapiens
APEFLGGPSVFLFPPKPKDTLMISRTPEVTCVVVDVSQEDPEVQFNWYVDGVEVHNAKTKPREEQFNSTYRVVSVLTVLHQDWLSGKEYKCKVSSKGLPSSIEKTISNATGQPREPQVYTLPPSQEEMTKNQVSLTCLVKGFYPSDIAVEWESNGQPENNYKTTPPVLDSDGSFFLYSRLTVDKSSWQEGNVFSCSVMHEALHNHYTQKSLSLSLGK

(19) United States
(12) Patent Application Publication
(10) Pub. No.: US 2021/0170025 A1
(43) Pub. Date: Jun. 10, 2021

(54) GP96-BASED CANCER THERAPY

(71) Applicant: Heat Biologics, Inc., Durham, NC (US)

(72) Inventors: Jeff HUTCHINS, Durham, NC (US); Lori MCDERMOTT, Durham, NC (US)

(21) Appl. No.: 16/763,867

(22) PCT Filed: Nov. 27, 2018
(86) PCT No.: PCT/US2018/062621

§ 371 Date: May 13, 2020

Related U.S. Application Data
(60) Provisional application No. 62/590,785, filed on Nov. 27, 2017.
(60) Provisional application No. 62/635,958, filed on Feb. 27, 2018.

(51) Int. Cl. A61K 39/395; A61P 35/00; C07K 16/28; A61K 39/00

(52) U.S. Cl. CPC A61K 39/39558; A61P 35/00; C07K 16/2818; C07K 16/2827; A61K 2039/86; A61K 2039/55516; A61K 2039/545; A61K 2039/505; A61K 2039/5156; A61K 39/001176

(57) ABSTRACT

The present disclosure relates, inter alia, to compositions and methods for treating cancer, including lung cancer (e.g., Non-Small Cell Lung Cancer), comprising administering (a) a cell harboring an expression vector comprising a nucleotide sequence that encodes a secretable vaccine protein and (b) an immune checkpoint inhibitor to a subject in need thereof.

### CROSS-REFERENCE TO RELATED APPLICATIONS

This application claims priority to and the benefit of U.S. Provisional patent Application No. 62/590,785, filed on Nov. 27, 2017, and U.S. Provisional patent Application No. 62/635,958, filed on Feb. 27, 2018, the entire contents of which are herein incorporated by reference herein in their entireties.

### FIELD OF THE DISCLOSURE

The present disclosure relates to compositions and methods for treating cancer, including lung cancer (e.g., Non-Small Cell Lung Cancer).

### DESCRIPTION OF THE TEXT FILE SUBMITTED ELECTRONICALLY

The contents of the text file submitted electronically herewith are incorporated herein by reference in their entirety: A computer readable format copy of the Sequence Listing (filename: HTB-027PC_Sequence Listing_ST25; date recorded: Oct. 23, 2018; file size: 18.8 KB).

### BACKGROUND

Cancer is a significant health problem worldwide. Despite recent advances that have been made in detection and therapy of cancer, no vaccine or other universally successful method for prevention or treatment is currently available. Current therapies, which are generally based on a combination of chemotherapy or surgery and radiation, continue to prove inadequate in many patients.

Lung cancer is the major cause of cancer death in the US, resulting in more than 1.4 million deaths per year. Early detection is difficult since clinical symptoms are delayed until the disease has reached an advanced stage. Current diagnostic methods include chest x-rays and the analysis of the type of cells contained in sputum and fiberoptic examination of the bronchial passages. Treatment regimens are determined by the type and stage of the cancer, and include surgery, radiation therapy and/or chemotherapy. In spite of considerable research into therapies for lung cancer and other cancers, lung cancer remains difficult to diagnose and treat effectively.

Accordingly, there exists a need in the art for improved methods for treating and preventing the recurrence of cancers, especially, lung cancer in patients. The present disclosure fulfills these needs and further provides other related advantages.

### SUMMARY OF THE INVENTION

The present disclosure relates, in some aspects, to methods for activation of CD8+ T cells to turn “cold” tumors into “hot” tumors, e.g., lung tumors using a cell-based, gp96-comprising vaccine. Accordingly, in various aspects, the present methods provide tumor T cell modulation such that tumors, e.g., lung tumors, are more to susceptible to anti-present methods provide tumor T cell modulation such that tumors, e.g., lung tumors, are more to susceptible to anti-tumor therapies, e.g. checkpoint inhibition therapies. Therefore, in various embodiments, the present methods provide for an expansion of the percentage of patients responding to checkpoint inhibitors or the conversion of a patient non-responding checkpoint inhibition to a responder (e.g. as an adjuvant or neoadjuvant).

In one aspect of the method of the disclosure, treating lung cancer, comprises administering (a) a cell harboring an expression vector comprising a nucleotide sequence that encodes a secretable vaccine protein and (b) an immune checkpoint inhibitor to a subject in need thereof. In some embodiments, the immune checkpoint inhibitor, inhibits an immune checkpoint gene. In some embodiments, the immune checkpoint inhibitor comprises an antibody or antigen binding fragment thereof.

In some embodiments, the immune check point inhibitor is an anti-PD-1 antibody or antigen binding fragment thereof. In some embodiments, the immune check point inhibitor is an anti-PD-L1 antibody or antigen binding fragment thereof.

In some embodiments, the anti-PD-1 or PD-L1 antibody or antigen binding fragment thereof is nivolumab, pembrolizumab, pidilizumab, BMS-936559, atezolizumab or avelumab. In some embodiments, the anti-PD-1 antibody is selected from nivolumab and pembrolizumab. In some embodiments, the anti-PD-1 antibody is Nivolumab. In some embodiments, the anti-PD-L1 antibody is durvalumab.

In some embodiments of the methods of the disclosure, the lung cancer is a small cell lung cancer. In some embodiments, the lung cancer is a Non-small cell lung cancer. In some embodiments, the Non-small cell lung cancer is adenocarcinoma. In some embodiments, the Non-small cell lung cancer is squamous cell carcinoma or large cell lung cancer.

In some embodiments of the method of the disclosure, the method reduces lung cancer recurrence. In some embodiments, the method increases the activation or proliferation of tumor antigen specific T cells in the subject. In some embodiments, the method increases the activation or the number of IFN-γ secreting CD8+ T cells in the subject.

In embodiments, the present methods include specific treatment regiment, such as, by way of illustration, a weekly dose of HS-110 for at least 16 weeks and a biweekly dose of an anti-PD-1 antibody for at least 16 weeks or a weekly dose of HS-110 for at least 6 weeks and a biweekly dose of an anti-PD-1 antibody for at least 6 weeks. In embodiments, the present methods are efficacious in patient populations that are not satisfactorily responsive to monotherapy with an anti-PD-1 antibody, such as patients who are PD-L1^{negative} or PD-L1^{low} or patients who have low tumor infiltrating lymphocytes (TILs) status (TIL^{low}).

In some embodiments, the subject exhibits a robust increase in immune response following administration. In some embodiments, the robust increase in immune response is defined as an increase of at least 2 fold above the baseline in the activation or proliferation of CD8+ T cells. In some embodiments, the CD8+ T cells secrete IFN-γ. In some embodiments, the method is more effective in reducing lung cancer recurrence in the subject compared to a subject who does not exhibit a robust increase in immune response. In some embodiments, the subject exhibits a low number of tumor infiltrating lymphocytes (TILs) prior to administration. In some embodiments, the method is more effective in reducing cancer recurrence or progression in the subject as compared to treatment with the immune checkpoint inhibitor alone.

In some aspects of the method of the disclosure, the vector is a mammalian expression vector. In some embodiments, the vaccine protein is a secretable gp96-Ig fusion protein which optionally lacks the gp96 KDEL (SEQ ID NO:3) sequence. In some embodiments, the Ig tag in the gp96-Ig fusion protein comprises the Fc region of human IgG1, IgG2, IgG3, IgG4, IgM, IgA, or IgE. In some embodiments, the expression vector comprises DNA. In some embodiments, the expression vector comprises RNA.

In some embodiments, the cell is a human tumor cell. In some embodiments, the cell is an irradiated or live and attenuated human tumor cell. In some embodiments, the human tumor cell is a cell from an established NSCLC, bladder cancer, melanoma, ovarian cancer, renal cell carcinoma, prostate carcinoma, sarcoma, breast carcinoma, squamous cell carcinoma, head and neck carcinoma, hepatocellular carcinoma, pancreatic carcinoma, or colon carcinoma cell line. In some embodiments, the human tumor cell line is a NSCLC cell line.

In some embodiments, prior to the administering of (a) the cell harboring the expression vector comprising the nucleotide sequence that encodes the secretable vaccine protein, and prior to the administering of (b) the immune checkpoint inhibitor, the subject has experienced disease progression after receiving a therapy. In some embodiments, the therapy is an immune checkpoint inhibitor therapy. In some embodiments, the therapy comprises chemotherapy. In some embodiments, the subject is a poor responder to the immune checkpoint inhibitor therapy. In some embodiments, the subject has failed the immune checkpoint inhibitor therapy. In some embodiments, the disease in the subject has progressed even when administered the immune checkpoint inhibitor therapy.

In embodiments, the patient has experienced disease progression after receiving a therapy. In embodiments, the therapy is an immune checkpoint inhibitor therapy. In embodiments, the therapy comprises chemotherapy. In embodiments, the patient is a poor responder to the immune checkpoint inhibitor therapy. In embodiments, the patient has failed the immune checkpoint inhibitor therapy. In embodiments, the disease in the patient has progressed even when administered the immune checkpoint inhibitor therapy.

### BRIEF DESCRIPTION OF THE DRAWINGS

FIG. 1A is a non-limiting schematic demonstrating a Phase 1b/2 study of Viagenpumatucel-L (HS-110) in combination with multiple treatment regimens in patients with non-small cell lung cancer (The “DURGA” Trial). FIG. 1B is an overview of the HS110-102 DURGA Trial Patient Population, and FIG. 10 is an overview of the DURGA Trial design.

FIG. 2 is a non-limiting schematic demonstrating a clinical trial design. Briefly patients with advanced and previously treated lung cancer were treated weekly with viagenpumatucel-L (HS-110) for 18 weeks and nivolumab 3 mg/kg every 2 weeks until disease progression or death. Biopsy tissue at baseline and at week 10 were tested for levels of CD8+ TILs and PD-L1 expression on tumor cells. Peripheral blood was analyzed for immunologic response using the Enzyme-Linked ImmunoSpot (ELISPOT) assay at weeks 1, 4, 7, 13 and at the end of HS-110 treatment.

FIG. 3A is a summary of the primary efficacy analysis in the Intention to Treat (ITT), Per Protocol (PP), and Completer populations. FIG. 3B is a table showing 5 RECIST responses (RECIST 1.1) for checkpoint inhibitor (CPI) naïve ITT patients (left column); (CR=complete response; PR=partial response; SD=stable disease; PD=progressive disease; and NE=not evaluable), the number of CPI ITT patients (middle column), and the percentage of the objective response rate (ORR).

FIG. 4 is a bar graph showing the best target lesion response by RECIST 1.1 in the per protocol population. The bar graph shows all evaluable ITT patients (Cohort A) with a baseline and on-treatment scan (n=38).

FIG. 5 is a line graph showing the durability of target lesion response in the per protocol (PP) population (Cohort A).

FIG. 6 is a survival plot showing overall percent survival data in the ITT patient population (Cohort A).

FIG. 7 is a survival plot showing a trend of improved survival with treatment duration in the completer population (Cohort A). The top curve is 16+ doses, and bottom curve is <16 doses.

FIG. 8 is a survival plot showing an overall percent survival by tumor infiltrating lymphocyte (TIL) level at baseline for high TIL (>10%) patients and low TIL (≤10%) patients for the checkpoint inhibitor (CPI) naïve population (Cohort A). The top curve at day 800 is Low TIL and the bottom curve is High TIL.

FIG. 9A and FIG. 9B are graphs showing progression free survival (PFS) in the CPI naïve ITT population (FIG. 9A; Cohort A), and the progression free survival by TIL level at baseline (FIG. 9B; Cohort A) in the CPI naïve ITT population. In FIG. 9A, the mean PFS (mPFS) was 58 days and the 1 year PFS was 23.9%. In FIG. 9B, the 1 year PFS for low TILs was 31.7%, and the 1 year PFS for high TILs was 10.6%. In FIG. 9B, the top curve at day 400 is Low TIL and the bottom curve is High TIL.

FIG. 10A-B are a pair of bar graphs showing the best target lesion response based on Tumor infiltrating lymphocytes (TIL) status in the per protocol population, which was CPI naïve (Cohort A). FIG. 10A shows the change from baseline in ≤10% CD8+ TIL and FIG. 10B shows the change from baseline in >10% CD8+ TIL.

FIG. 11 shows two line graphs depicting the durability of target lesion response based on TIL Status in the per protocol population (Cohort A). In FIG. 11, the “high” response is represented by the dashed line, and the “Low” response is represented by the solid line.

FIG. 12 is a pair of bar graphs showing best target lesion response based on PD-L1 Status (Cohort A). FIG. 12, right panel shows the change from baseline in >1% PD-L1 tumor type in the per protocol population. FIG. 12, left panel shows the change from baseline in <1% PD-L1 tumor type in the per protocol population.

FIG. 13 is a pair of line graphs showing the durability of target lesion response based on PD-L1 status in the per protocol population (Cohort A). FIG. 13 shows the change from baseline in >1% PD-L1 tumor type, and shows the change from baseline in <1% PD-L1 tumor type. In FIG. 13, the “(−)ive” response is represented by the dashed line, and the “ ”(±)ive” response is represented by the solid line.

FIG. 14 is a bar graph showing a Completer Analysis for the best target lesion response activity (Cohort A). It depicts each patient who completed study treatment with viagenpumatucel-L and plots the percent change in tumor lesion size from baseline to their best assessment per RECIST 1.1. Dotted lines represent the RECIST 1.1 cut-offs for progressive disease (PD, >20% increase in sum of the longest diameters [SLD]), stable disease (SD, <20% increase and <30% decrease in SLD) and partial response (PR, >30% decrease in SLD). A positive ELISPOT response was determined to be a ≥2-fold increase in δ-INF driven activity over baseline.

FIG. 15 is a plot showing the ELISPOT activity and survival (Cohort A). High=ELISPOT activity above the median of patients tested. Low=ELISPOT activity below the median of patients tested.

FIG. 16 is plot showing that ELISPOT response correlates with long-term and overall survival (Cohort A). Setting the alpha probability at 0.1 the number of ELISPOT spots generated from stimulating patient PBMCs with whole cell HS110 vaccine lysates correlates (p=0.06) significantly with the overall survival of patients on therapy.

FIG. 17A and FIG. 17B are graphs showing the percentage of CPI naïve patients who experienced progression free survival (PFS) (FIG. 17A), or who experienced Overall Survival (OS) (FIG. 17B), by PD-L1 level at baseline (Cohort A). In FIG. 17A, at day 200, the top curve (solid line) is PD-L1 and the bottom curve (dashed line) is PD-L1. In FIG. 17B, at day 414, the top curve (dashed line) is PD-L1 and the bottom curve (solid line) is PD-L1.

FIG. 18 is a survival plot showing overall percent survival data in the progression free survival (PFS) ITT patient population (Cohort B). In this patient population, the patients previously received CPI therapy, however, disease progressed after 6 months or longer. By “censoring” is meant patients lost to follow-up, a recognized data management tool.

FIG. 19 is a table showing 5 RECIST responses (RECIST 1.1) for checkpoint inhibitor (CPI) progressor ITT patients (left column); (CR=complete response; PR=partial response; SD=stable disease; PD=progressive disease; and NE=not evaluable), the number of CPI ITT patients (middle column), and the percentage of the objective response rate (ORR) (Cohort B).

FIG. 20 is a bar graph showing the best target lesion response activity for checkpoint inhibitor (CPI) progressor ITT patients (Cohort B).

FIG. 21 is a line graph showing the durability of target lesion response for the CPI progressor ITT patient population (Cohort B).

FIG. 22 shows two bar graphs depicting the best target lesion response based on Tumor infiltrating lymphocytes (TIL) status in the checkpoint inhibitor (CPI) progressor population (Cohort B). The bar graph on the left side of FIG. 22 shows the change from baseline in ≤10% CD8+ TIL (low TIL) and the bar graph on the right side of FIG. 22 shows the change from baseline in >10% CD8+ TIL (high TIL).

FIG. 23 shows two line graphs depicting the durability of target lesion response based on TIL level in the CPI progressor population (Cohort B). In FIG. 23, the “high” response is represented by the dashed line, and the “low” response is represented by the solid line.

FIG. 24 shows two bar graphs depicting the best target lesion response based on PD-L1 Status in the CPI progressor population (Cohort B). The bar graph on the left side of FIG. 24 shows the change from baseline in <1% PD-L1 tumor type for the CPI progressor population. The bar graph on the right side of FIG. 24 shows the change from baseline in ≥1% PD-L1 tumor type for the CPI progressor population.

FIG. 25 is a pair of line graphs showing the durability of target lesion response based on PD-L1 status in the CPI progressor population (Cohort B). FIG. 25 shows the change from baseline in >1% PD-L1 tumor type, and shows the change from baseline in <1% PD-L1 tumor type. In FIG. 25, the “(−)ive” response is represented by the dashed line, and the “ ”(+)ive” response is represented by the solid line.

### DETAILED DESCRIPTION OF THE DISCLOSURE

The present disclosure is based on the discovery that a combination vaccine therapy involving a low dose amount of a cell line that expresses a modified and secretable heat shock protein (i.e., gp96-Ig) and an immune checkpoint inhibitor (e.g., anti-PD-1 antibody or antigen binding fragment thereof) is particularly effective for treating lung cancer, including Non-Small Cell Lung Cancer (NSCLC). In some embodiments, the present methods synergistically activate immune responses against tumor cells resulting in reduced lung cancer recurrence and improved survival.

Immunosuppression may develop in (NSCLC) patients in a variety of ways, such as activation of checkpoint pathways in the tumor microenvironment. Drugs that disrupt checkpoint molecule signaling like anti-PD-1 monoclonal antibodies may release this brake on the immune system. Tumor expression of PD-L1, the ligand of PD-1, plays an important role in patient response to checkpoint inhibitors; in general, clinical response to checkpoint inhibitors requires tumor expression of PD-L1 and the presence of Tumor Infiltrating Lymphocytes (TILs).

Viagenpumatucel-L is a proprietary, allogeneic tumor cell vaccine expressing a recombinant secretory form of the heat shock protein gp96 fusion (gp96-Ig) with potential antineoplastic activity. Upon administration of viagenpumatucel-L, irradiated live tumor cells continuously secrete gp96-Ig along with its chaperoned tumor associated antigens (TAAs) into the dermal layers of the skin, thereby activating antigen presenting cells, natural killer cells and priming potent cytotoxic T lymphocytes (CTLs) to respond against TAAs presented on the endogenous tumor cells. Furthermore, Viagenpumatucel-L induces long-lived memory T cells that can fight recurring cancer cells.

The present invention is directed to the finding that co-administration of Viagenpumatucel-L with anti-PD-1 agents enhances the vaccine's anti-tumor activity while prolonging or increasing the efficacy of the checkpoint inhibitor, creating a synergistic effect. This surprising effect is seen even in patients that have low PD-L1 status (e.g. their tumors do not exhibit high levels of PD-L1 (PD-L1^{high} but rather are PD-L1^{negative} or PD-L1^{low} as described herein). That is, the addition of the VIAGENPUMATUCEL-L composition surprisingly allows even patients who would not normally be treated with an anti-PD-1 antibody to exhibit clinical benefits.

Furthermore, as is more fully described herein, patients who have “cold” tumors, e.g. that have low amounts of CD8+ TILs, are not generally very responsive to anti-PD-1 antibodies, generally exhibiting about a 10% response rate with nivolumab alone; see Teng et al. Cancer Research 75(11): Jun. 1, 2015. However, the combination therapies outlined herein surprisingly are equally as effective irrespective of the TIL status of the patient, showing that the Viagenpumatucel-L expands anti-PD-1 therapeutic efficacy in TIL^{low} patients.

Accordingly, the present invention provides combination therapies of Viagenpumatucel-L and anti-PD-1 antibodies to treat patients with NSCLC.

The present invention provides methods of treating cancer, particularly Non Small Cell Lung Cancer (“NSCLC”), by co-administering Viagenpumatucel-L in combination with an anti-PD-1 antibody. As will be understood by one of skill in the art, “co-administration” in this context means that the patient receives doses of Viagenpumatucel-L as well as doses of an anti-PD-1 antibody during the time course of treatment. In general, these therapies are delivered by separate routes of administration to the patient, rather than as a mixture, particularly as the anti-PD-1 antibody is generally delivered less frequently than the Viagenpumatucel-L doses.

The present invention provides combinations of Viagenpumatucel-L and an anti-PD-1 antibody. Viagenpumatucel-L (sometimes also referred to herein as “HS-110”) is a cellular composition comprising a vector that encodes a fusion protein, gp96-Ig, described herein. The heat shock protein (hsp) gp96 serves as a chaperone for peptides on their way to MHC class I molecules expressed on antigen-presenting or dendritic cell. Gp96 obtained from tumor cells and used as a vaccine can induce specific tumor immunity, presumably through the transport of tumor-specific peptides to antigen-presenting cells (APCs) (J Immunol 1999, 163(10):5178-5182). For example, gp96-associated peptides are cross-presented to CD8 cells by dendritic cells (DCs) upon uptake of the scavenger receptor (CD91).

Accordingly, the present invention provides cells comprising vectors that encode gp-96-Ig fusion proteins.

The Viagenpumatucel-L compositions of the invention include vectors that encode gp-96-Ig fusion proteins. Thus, the vectors provided herein contain a nucleotide sequence that encodes a gp96-Ig fusion protein. The coding region of human gp96 is 2,412 bases in length (SEQ ID NO:1), and encodes an 803 amino acid protein (SEQ ID NO:2) which includes a 21 amino acid signal peptide at the amino terminus, a potential transmembrane region rich in hydrophobic residues, and an ER retention peptide sequence at the carboxyl terminus (GENBANK Accession No. X15187; see Maki et al, Proc Natl Acad Sci USA 1990, 87:5658-5562).

An exemplary nucleic acid sequence encoding the human gp96 gene, the KDEL deletion, and the nucleotide sequence are shown in SEQ ID NO: 4. Additionally, as noted herein, the last 4 amino acids of gp96, “KDEL” is deleted as discussed herein. KDEL is a retention sequence that normally serves as an endoplasmatic reticulum-resident chaperone peptide, and the present invention relies on secretable gp96 fusion proteins as discussed herein.

In some embodiments, the gp96 portion of a gp96-Ig fusion protein can contain all or a portion of a wild type gp96 sequence (e.g., the human sequence set forth in SEQ ID NO:2). For example, a secretable gp96-Ig fusion protein can include the first 799 amino acids of SEQ ID NO:2, such that it lacks the C-terminal KDEL (SEQ ID NO:3; the amino acid sequence without the endoplasmic retention sequence is shown as SEQ ID NO:4).

Additionally, the gp96 portion of the fusion protein can have an amino acid sequence that contains one or more substitutions, deletions, or additions as compared to the first 799 amino acids of the wild type gp96 sequence, such that it has at least 90% (e.g., at least 90%, at least 91%, at least 92%, at least 93%, at least 94%, at least 95%, at least 96%, at least 97%, at least 98%, or at least 99%) sequence identity to the wild type polypeptide.

“Percent (%) amino acid sequence identity” with respect to a protein sequence is defined as the percentage of amino acid residues in a candidate sequence that are identical with the amino acid residues in the specific (parental) sequence, after aligning the sequences and introducing gaps, if necessary, to achieve the maximum percent sequence identity, and not considering any conservative substitutions as part of the sequence identity. Alignment for purposes of determining percent amino acid sequence identity can be achieved in various ways that are within the skill in the art, for instance, using publicly available computer software such as BLAST, BLAST-2, ALIGN or Megalign (DNASTAR) software. Those skilled in the art can determine appropriate parameters for measuring alignment, including any algorithms needed to achieve maximal alignment over the full length of the sequences being compared. One particular program is the ALIGN-2 program outlined at paragraphs [0279] to [0280] of US Pub. No. 20160244525, hereby incorporated by reference. Another approximate alignment for nucleic acid sequences is provided by the local homology algorithm of Smith and Waterman, Advances in Applied Mathematics, 2:482-489 (1981). This algorithm can be applied to amino acid sequences by using the scoring matrix developed by Dayhoff, Atlas of Protein Sequences and Structure, M. O. Dayhoff ed., 5 suppl. 3:353-358, National Biomedical Research Foundation, Washington, D.C., USA, and normalized by Gribskov, Nucl. Acids Res. 14(6):6745-6763 (1986).

An example of an implementation of this algorithm to determine percent identity of a sequence is provided by the Genetics Computer Group (Madison, Wis.) in the “BestFit” utility application. The default parameters for this method are described in the Wisconsin Sequence Analysis Package Program Manual, Version 8 (1995) (available from Genetics Computer Group, Madison, Wis.). Another method of establishing percent identity in the context of the present invention is to use the MPSRCH package of programs copyrighted by the University of Edinburgh, developed by John F. Collins and Shane S. Sturrok, and distributed by IntelliGenetics, Inc. (Mountain View, Calif.). From this suite of packages, the Smith-Waterman algorithm can be employed where default parameters are used for the scoring table (for example, gap open penalty of 12, gap extension penalty of one, and a gap of six). From the data generated the “Match” value reflects “sequence identity.” Other suitable programs for calculating the percent identity or similarity between sequences are generally known in the art, for example, another alignment program is BLAST, used with default parameters. For example, BLASTN and BLASTP can be used using the following default parameters: genetic code=standard; filter=none; strand=both; cutoff=60; expect=10; Matrix=BLOSUM62; Descriptions=50 sequences; sort by=HIGH SCORE; Databases=non-redundant, GenBank+EMBL+DDBJ+PDB+GenBank CDS translations+Swiss protein+Spupdate+PIR. Details of these programs can be found at the internet address located by placing http:// in front of blast.ncbi.nlm.nih.gov/Blast.cgi.

The degree of identity between an amino acid sequence of the present invention (“invention sequence”) and the parental amino acid sequence is calculated as the number of exact matches in an alignment of the two sequences, divided by the length of the “invention sequence,” or the length of the parental sequence, whichever is the shortest. The result is expressed in percent identity.

Thus, in some embodiments, the gp96 component of the nucleic acid encoding a gp96-Ig fusion polypeptide as described below can encode an amino acid sequence that differs from the wild type gp96 polypeptide at one or more amino acid positions.

The Viagenpumatucel-L compositions of the invention utilize the gp-96 as a fusion protein, gp-96-Ig. As described herein, gp96-Ig is constructed by replacing the KDEL retention sequence of gp96, normally an endoplasmatic reticulum-resident chaperone peptide, with the Fc portion of human IgG1, using an optional linker. As used herein, the Fc portion of human IgG1 include the CH2-CH3 domains and can optionally include the hinge region at the N-terminus (hinge-CH2-CH3). The sequence of the Fc domain absent the hinge is shown in SEQ ID NO: 5. In some cases, the IgG1 hinge serves as the linker joining the gp96 protein and the Fc domain.

In some embodiments, the vector comprising the gp96-Ig fusion protein comprises a linker. In various embodiments, the linker may be derived from naturally-occurring multi-domain proteins or are empirical linkers as described, for example, in Chichili et al., (2013), Protein Sci. 22(2):153-167, Chen et al, (2013), Adv Drug Deliv Rev. 65(10):1357-1369, the entire contents of which are hereby incorporated by reference. In some embodiments, the linker may be designed using linker designing databases and computer programs such as those described in Chen et al., (2013), Adv Drug Deliv Rev. 65(10):1357-1369 and Crasto et. al., (2000), Protein Eng. 13(5):309-312, the entire contents of which are hereby incorporated by reference.

In some embodiments, the linker is a synthetic linker such as PEG. In some embodiments, the linker is a polypeptide. In some embodiments, the linker is less than about 100 amino acids long. For example, the linker may be less than about 100, about 95, about 90, about 85, about 80, about 75, about 70, about 65, about 60, about 55, about 50, about 45, about 40, about 35, about 30, about 25, about 20, about 19, about 18, about 17, about 16, about 15, about 14, about 13, about 12, about 11, about 10, about 9, about 8, about 7, about 6, about 5, about 4, about 3, or about 2 amino acids long. In some embodiments, the linker is flexible. In another embodiment, the linker is rigid. In various embodiments, the linker is substantially comprised of glycine and serine residues (e.g., about 30%, or about 40%, or about 50%, or about 60%, or about 70%, or about 80%, or about 90%, or about 95%, or about 97% glycines and serines).

In some embodiments, the linker is a hinge region of an antibody (e.g., of IgG, IgA, IgD, and IgE, inclusive of subclasses (e.g. IgG1, IgG2, IgG3, and IgG4, and IgA1 and IgA2)). The hinge region, found in IgG, IgA, IgD, and IgE class antibodies, acts as a flexible spacer, allowing the Fab portion to move freely in space. In contrast to the constant regions, the hinge domains are structurally diverse, varying in both sequence and length among immunoglobulin classes and subclasses. For example, the length and flexibility of the hinge region varies among the IgG subclasses. The hinge region of IgG1 encompasses amino acids 216-231 and, because it is freely flexible, the Fab fragments can rotate about their axes of symmetry and move within a sphere centered at the first of two inter-heavy chain disulfide bridges. IgG2 has a shorter hinge than IgG1, with 12 amino acid residues and four disulfide bridges. The hinge region of IgG2 lacks a glycine residue, is relatively short, and contains a rigid poly-proline double helix, stabilized by extra inter-heavy chain disulfide bridges. These properties restrict the flexibility of the IgG2 molecule. IgG3 differs from the other subclasses by its unique extended hinge region (about four times as long as the IgG1 hinge), containing 62 amino acids (including 21 prolines and 11 cysteines), forming an inflexible poly-proline double helix. In IgG3, the Fab fragments are relatively far away from the Fc fragment, giving the molecule a greater flexibility. The elongated hinge in IgG3 is also responsible for its higher molecular weight compared to the other subclasses. The hinge region of IgG4 is shorter than that of IgG1 and its flexibility is intermediate between that of IgG1 and IgG2. The flexibility of the hinge regions reportedly decreases in the order IgG3>IgG1>IgG4>IgG2.

Additional illustrative linkers include, but are not limited to, linkers having the sequence LE, GGGGS, (GGGGS)n (n=1-4), (Gly)8, (Gly)6, (EAAAK)n (n=1-3), A(EAAAK)nA (n=2-5), AEAAAKEAAAKA, A(EAAAK)4ALEA(EAAAK)4A, PAPAP, KESGSVSSEQLAQFRSLD, EGKSSGSGSESKST, GSAGSAAGSGEF, and (XP)n, with X designating any amino acid, e.g., Ala, Lys, or Glu.

In some embodiments, the linker may be functional. For example, without limitation, the linker may function to improve the folding and/or stability, improve the expression, improve the pharmacokinetics, and/or improve the bioactivity of the present compositions. In another example, the linker may function to target the compositions to a particular cell type or location.

In some embodiments, a gp96 peptide can be fused to the hinge, CH2 and CH3 domains of murine IgG1 (Bowen et al, J Immunol 1996, 156:442-449). This region of the IgG1 molecule contains three cysteine residues that normally are involved in disulfide bonding with other cysteines in the Ig molecule. Since none of the cysteines are required for the peptide to function as a tag, one or more of these cysteine residues can be substituted by another amino acid residue, such as, for example, serine.

In some embodiments, the present disclosure provides a vector encoding a modified and secretable heat shock protein (i.e., gp96-Ig). A nucleic acid encoding a gp96-Ig fusion sequence can be produced using the methods described in U.S. Pat. No. 8,685,384, which is incorporated herein by reference in its entirety.

DNAs encoding immunoglobulin light or heavy chain constant regions are known or readily available from cDNA libraries. See, for example, Adams et al, Biochemistry 1980, 19:2711-2719; Gough et al., Biochemistry 1980 19:2702-2710; Dolby et al, Proc Natl Acad Sci USA 1980, 77:6027-6031; Rice et al., Proc Natl Acad Sci USA 1982, 79:7862-7865; Falkner et al., Nature 1982, 298:286-288; and Morrison et al., Ann Rev Immunol 1984, 2:239-256. Since many immunological reagents and labeling systems are available for the detection of immunoglobulins, gp96-Ig fusion proteins can readily be detected and quantified by a variety of immunological techniques known in the art, such as enzyme-linked immunosorbent assay (ELISA), immunoprecipitation, and fluorescence activated cell sorting (FACS). Similarly, if the peptide tag is an epitope with readily available antibodies, such reagents can be used with the techniques mentioned above to detect, quantitate, and isolate gp96-Ig fusions.

Various leader sequences known in the art also can be used for efficient secretion of gp96-Ig fusion proteins from bacterial and mammalian cells (see, von Heijne, J Mol Biol 1985, 184:99-105). Leader peptides can be selected based on the intended host cell, and may include bacterial, yeast, viral, animal, and mammalian sequences. For example, the herpes virus glycoprotein D leader peptide is suitable for use in a variety of mammalian cells. Another leader peptide for use in mammalian cells can be obtained from the V-J2-C region of the mouse immunoglobulin kappa chain (Bernard et al, Proc Natl Acad Sci USA 1981, 78:5812-5816). DNA sequences encoding peptide tags or leader peptides are known or readily available from libraries or commercial suppliers, and are suitable in the fusion proteins described herein.

Furthermore, in some embodiments, one may substitute the gp96 of the present disclosure with one or more vaccine proteins. For instance, various heat shock proteins are among the vaccine proteins. In various embodiments, the heat shock protein is one or more of a small hsp, hsp40, hsp60, hsp70, hsp90, and hsp110 family member, inclusive of fragments, variants, mutants, derivatives or combinations thereof (Hickey, et al., 1989, Mol. Cell. Biol. 9:2615-2626; Jindal, 1989, Mol. Cell. Biol. 9:2279-2283).

In some embodiments, the present disclosure provides nucleic acid constructs that encode a vaccine protein fusion protein (e.g., a gp96-Ig fusion protein) that can be expressed in prokaryotic and eukaryotic cells. For example, the present disclosure provides expression vectors (e.g., DNA- or RNA-based vectors) containing nucleotide sequences that encode a vaccine protein fusion (e.g., a gp96-Ig fusion). In addition, the present invention provides methods for making the vectors described herein, as well as methods for introducing the vectors into appropriate host cells for expression of the encoded polypeptides. In general, the methods provided herein include constructing nucleic acid sequences encoding a vaccine protein fusion protein (e.g., a gp96-Ig fusion protein) and cloning the sequences encoding the fusion proteins into an expression vector. The expression vector can be introduced into host cells, either of which can be administered to a subject to, for example, treat cancer. For example, the gp96-Ig based vaccines can be generated to stimulate antigen specific immune responses against tumor antigens.

In some embodiments, cDNA or DNA sequences encoding the vaccine protein fusion (e.g., a gp96-Ig fusion) can be obtained (and, if desired, modified) using conventional DNA cloning and mutagenesis methods, DNA amplification methods, and/or synthetic methods. In general, a sequence encoding a vaccine protein fusion protein (e.g., a gp96-Ig fusion protein) can be inserted into a cloning vector for genetic modification and replication purposes prior to expression. Each coding sequence can be operably linked to a regulatory element, such as a promoter, for purposes of expressing the encoded protein in suitable host cells in vitro and in vivo.

Both prokaryotic and eukaryotic vectors can be used for expression of the vaccine protein (e.g., gp96-Ig) in the methods provided herein. Prokaryotic vectors include constructs based on E. coli sequences (see, e.g., Makrides, Microbiol Rev 1996, 60:512-538). Non-limiting examples of regulatory regions that can be used for expression in E. coli include lac, trp, Ipp, phoA, recA, tac, T3, T7 and APL. Non-limiting examples of prokaryotic expression vectors may include the λgt vector series such as λgt11 (Huynh et al, in “DNA Cloning Techniques, Vol. I: A Practical Approach,” 1984, (D. Glover, ed.), pp. 49-78, IRL Press, Oxford), and the pET vector series (Studier et al, Methods Enzymol 1990, 185:60-89). Prokaryotic host-vector systems cannot perform much of the post-translational processing of mammalian cells, however. Thus, eukaryotic host-vector systems may be particularly useful.

A variety of regulatory regions can be used for expression of the vaccine protein (e.g., gp96-Ig) and T cell costimulatory fusions in mammalian host cells. For example, the SV40 early and late promoters, the cytomegalovirus (CMV) immediate early promoter, and the Rous sarcoma virus long terminal repeat (RSV-LTR) promoter can be used. Inducible promoters that may be useful in mammalian cells include, without limitation, promoters associated with the metallothionein II gene, mouse mammary tumor virus glucocorticoid responsive long terminal repeats (MMTV-LTR), the β-interferon gene, and the hsp70 gene (see, Williams et al, Cancer Res 1989, 49:2735-42; and Taylor et al, Mol Cell Biol 1990, 10:165-75). Heat shock promoters or stress promoters also may be advantageous for driving expression of the fusion proteins in recombinant host cells.

In one aspect, the present disclosure contemplates the use of inducible promoters capable of effecting high level of expression transiently in response to a cue. Illustrative inducible expression control regions include those comprising an inducible promoter that is stimulated with a cue such as a small molecule chemical compound. Particular examples can be found, for example, in U.S. Pat. Nos. 5,989,910, 5,935,934, 6,015,709, and 6,004,941, each of which is incorporated herein by reference in its entirety.

Animal regulatory regions that exhibit tissue specificity and have been utilized in transgenic animals also can be used in tumor cells of a particular tissue type: the elastase I gene control region that is active in pancreatic acinar cells (Swift et al., Cell 1984, 38:639-646; Ornitz et al., Cold Spring Harbor Symp Quant Biol 1986, 50:399-409; and MacDonald, Hepatology 1987, 7:425-515); the insulin gene control region that is active in pancreatic beta cells (Hanahan, Nature 1985, 315:115-122), the immunoglobulin gene control region that is active in lymphoid cells (Grosschedl et al., Cell 1984, 38:647-658; Adames et al., Nature 1985, 318:533-538; and Alexander et al., Mol Cell Biol 1987, 7:1436-1444), the mouse mammary tumor virus control region that is active in testicular, breast, lymphoid and mast cells (Leder et al., Cell 1986, 45:485-495), the albumin gene control region that is active in liver (Pinkert et al., Genes Devel, 1987, 1:268-276), the alpha-fetoprotein gene control region that is active in liver (Krumlauf et al., Mol Cell Biol 1985, 5:1639-1648; and Hammer et al., Science 1987, 235:53-58); the alpha 1-antitrypsin gene control region that is active in liver (Kelsey et al., Genes Devel 1987, 1:161-171), the beta-globin gene control region that is active in myeloid cells (Mogram et al., Nature 1985, 315:338-340; and Kollias et al., Cell 1986, 46:89-94); the myelin basic protein gene control region that is active in oligodendrocyte cells in the brain (Readhead et al., Cell 1987, 48:703-712); the myosin light chain-2 gene control region that is active in skeletal muscle (Sani, Nature 1985, 314:283-286), and the gonadotropic releasing hormone gene control region that is active in the hypothalamus (Mason et al., Science 1986, 234:1372-1378).

An expression vector also can include transcription enhancer elements, such as those found in SV40 virus, Hepatitis B virus, cytomegalovirus, immunoglobulin genes, metallothionein, and β-actin (see, Bittner et al., Meth Enzymol 1987, 153:516-544; and Gorman, Curr Op Biotechnol 1990, 1:36-47). In addition, an expression vector can contain sequences that permit maintenance and replication of the vector in more than one type of host cell, or integration of the vector into the host chromosome. Such sequences include, without limitation, to replication origins, autonomously replicating sequences (ARS), centromere DNA, and telomere DNA.

In addition, an expression vector can contain one or more selectable or screenable marker genes for initially isolating, identifying, or tracking host cells that contain DNA encoding fusion proteins as described herein. For long term, high yield production of gp96-Ig and T cell costimulatory fusion proteins, stable expression in mammalian cells can be useful. A number of selection systems can be used for mammalian cells. For example, the Herpes simplex virus thymidine kinase (Wigler et al., Cell 1977, 11:223), hypoxanthine-guanine phosphoribosyltransferase (Szybalski and Szybalski, Proc Natl Acad Sci USA 1962, 48:2026), and adenine phosphoribosyltransferase (Lowy et al., Cell 1980, 22:817) genes can be employed in tk−, hgprt−, or aprt− cells, respectively. In addition, antimetabolite resistance can be used as the basis of selection for dihydrofolate reductase (dhfr), which confers resistance to methotrexate (Wigler et al., Proc Natl Acad Sci USA 1980, 77:3567; O'Hare et al., Proc Natl Acad Sci USA 1981, 78:1527); gpt, which confers resistance to mycophenolic acid (Mulligan and Berg, Proc Natl Acad Sci USA 1981, 78:2072); neomycin phosphotransferase (neo), which confers resistance to the aminoglycoside G-418 (Colberre-Garapin et al., J Mol Biol 1981, 150:1); and hygromycin phosphotransferase (hyg), which confers resistance to hygromycin (Santerre et al., Gene 1984, 30:147). Other selectable markers such as histidinol and Zeocin™ also can be used.

A number of viral-based expression systems also can be used with mammalian cells to produce gp96-Ig. Vectors using DNA virus backbones have been derived from simian virus 40 (SV40) (Hamer et al, Cell 1979, 17:725), adenovirus (Van Doren et al., Mol Cell Biol 1984, 4:1653), adeno-associated virus (McLaughlin et al, J Virol 1988, 62:1963), and bovine papillomas virus (Zinn et al, Proc Natl Acad Sci USA 1982, 79:4897). When an adenovirus is used as an expression vector, the donor DNA sequence may be ligated to an adenovirus transcription/translation control complex, e.g., the late promoter and tripartite leader sequence. This fusion gene may then be inserted in the adenovirus genome by in vitro or in vivo recombination. Insertion in a non-essential region of the viral genome (e.g., region E1 or E3) can result in a recombinant virus that is viable and capable of expressing heterologous products in infected hosts. (See, e.g., Logan and Shenk, Proc Natl Acad Sci USA 1984, 81:3655-3659).

Bovine papillomavirus (BPV) can infect many higher vertebrates, including man, and its DNA replicates as an episome. A number of shuttle vectors have been developed for recombinant gene expression which exist as stable, multicopy (20-300 copies/cell) extrachromosomal elements in mammalian cells. Typically, these vectors contain a segment of BPV DNA (the entire genome or a 69% transforming fragment), a promoter with a broad host range, a polyadenylation signal, splice signals, a selectable marker, and “poisonless” plasmid sequences that allow the vector to be propagated in E. coli. Following construction and amplification in bacteria, the expression gene constructs are transfected into cultured mammalian cells by, for example, calcium phosphate coprecipitation. For those host cells that do not manifest a transformed phenotype, selection of transformants is achieved by use of a dominant selectable marker, such as histidinol and G418 resistance.

Alternatively, the vaccinia 7.5K promoter can be used. (See, e.g., Mackett et al., Proc Natl Acad Sci USA 1982, 79:7415-7419; Mackett et al., J Virol 1984, 49:857-864; and Panicali et al., Proc Natl Acad Sci USA 1982, 79:4927-4931.) In cases where a human host cell is used, vectors based on the Epstein-Barr virus (EBV) origin (OriP) and EBV nuclear antigen 1 (EBNA-1; a trans-acting replication factor) can be used. Such vectors can be used with a broad range of human host cells, e.g., EBO-pCD (Spickofsky et al., DNA Prot Eng Tech 1990, 2:14-18); pDR2 and ADR2 (available from Clontech Laboratories).

Gp96-Ig fusion proteins also can be made with retrovirus-based expression systems. Retroviruses, such as Moloney murine leukemia virus, can be used since most of the viral gene sequence can be removed and replaced with exogenous coding sequence while the missing viral functions can be supplied in trans. In contrast to transfection, retroviruses can efficiently infect and transfer genes to a wide range of cell types including, for example, primary hematopoietic cells. Moreover, the host range for infection by a retroviral vector can be manipulated by the choice of envelope used for vector packaging.

For example, a retroviral vector can comprise a 5′ long terminal repeat (LTR), a 3′ LTR, a packaging signal, a bacterial origin of replication, and a selectable marker. The gp96-Ig fusion protein coding sequence, for example, can be inserted into a position between the 5′ LTR and 3′ LTR, such that transcription from the 5′ LTR promoter transcribes the cloned DNA. The 5′ LTR contains a promoter (e.g., an LTR promoter), an R region, a U5 region, and a primer binding site, in that order. Nucleotide sequences of these LTR elements are well known in the art. A heterologous promoter as well as multiple drug selection markers also can be included in the expression vector to facilitate selection of infected cells. See, McLauchlin et al, Prog Nucleic Acid Res Mol Biol 1990, 38:91-135; Morgenstern et al., Nucleic Acid Res 1990, 18:3587-3596; Choulika et al, J Virol 1996, 70:1792-1798; Boesen et al., Biotherapy 1994, 6:291-302; Salmons and Gunzberg, Human Gene Ther 1993, 4:129-141; and Grossman and Wilson, Curr Opin Genet Devel 1993, 3:110-114.

Any of the cloning and expression vectors described herein may be synthesized and assembled from known DNA sequences using techniques that are known in the art. The regulatory regions and enhancer elements can be of a variety of origins, both natural and synthetic. Some vectors and host cells may be obtained commercially. Non-limiting examples of useful vectors are described in Appendix 5 of Current Protocols in Molecular Biology, 1988, ed. Ausubel et al., Greene Publish. Assoc. & Wiley Interscience, which is incorporated herein by reference; and the catalogs of commercial suppliers such as Clontech Laboratories, Stratagene Inc., and Invitrogen, Inc.

In some embodiments, the present disclosure utilizes a cell that is transfected with a vector encoding a gp96-Ig fusion protein. Without wishing to be bound by theory, it is believed that administration of gp96-Ig secreting cells triggers robust, antigen-specific CD8 cytotoxic T lymphocyte (CTL) expansion, combined with activation of the innate immune system. Tumor cell-secreted gp96 causes the recruitment of DCs and natural killer (NK) cells to the site of gp96 secretion, and mediates DC activation. Further, the endocytic uptake of gp96 and its chaperoned peptides triggers peptide cross presentation via major MHC class I, as well as strong, cognate CD8 activation independent of CD4 cells.

Accordingly, in various embodiments, the present invention further provides host cell lines that harbor a vector encoding a modified and secretable heat shock protein (e.g., gp96-Ig) as described herein. In some embodiments, the host cell line is administered to a subject for the treatment of lung cancer.

In some embodiments, expression vectors as described herein can be introduced into host cells for producing secreted vaccine proteins (e.g., gp96-Ig). There are a variety of techniques available for introducing nucleic acids into viable cells. Techniques suitable for the transfer of nucleic acid into mammalian cells in vitro include the use of liposomes, electroporation, microinjection, cell fusion, polymer-based systems, DEAE-dextran, viral transduction, the calcium phosphate precipitation method, etc. For in vivo gene transfer, a number of techniques and reagents may also be used, including liposomes; natural polymer-based delivery vehicles, such as chitosan and gelatin; viral vectors are also suitable for in vivo transduction. In some situations, it is desirable to provide a targeting agent, such as an antibody or ligand specific for a cell surface membrane protein. Where liposomes are employed, proteins which bind to a cell surface membrane protein associated with endocytosis may be used for targeting and/or to facilitate uptake, e.g., capsid proteins or fragments thereof tropic for a particular cell type, antibodies for proteins which undergo internalization in cycling, proteins that target intracellular localization and enhance intracellular half-life. The technique of receptor-mediated endocytosis is described, for example, by Wu et al, J. Biol. Chem. 262, 4429-4432 (1987); and Wagner et al., Proc. Natl. Acad. Sci. USA 87, 3410-3414 (1990).

Where appropriate, gene delivery agents such as, e.g., integration sequences can also be employed. Numerous integration sequences are known in the art (see, e.g., Nunes-Duby et al, Nucleic Acids Res. 26:391-406, 1998; Sadwoski, J. Bacteriol., 165:341-357, 1986; Bestor, Cell, 122(3):322-325, 2005; Plasterk et al, TIG 15:326-332, 1999; Kootstra et al, Ann. Rev. Pharm. Toxicol., 43:413-439, 2003). These include recombinases and transposases. Examples include Cre (Sternberg and Hamilton, J. Mol. Biol., 150:467-486, 1981), lambda (Nash, Nature, 247, 543-545, 1974), Flp (Broach, et al., Cell, 29:227-234, 1982), R (Matsuzaki, et al., J. Bacteriology, 172:610-618, 1990), cpC31 (see, e.g., Groth et al., J. Mol. Biol. 335:667-678, 2004), sleeping beauty, transposases of the mariner family (Plasterk et al., supra), and components for integrating viruses such as AAV, retroviruses, and antiviruses having components that provide for virus integration such as the LTR sequences of retroviruses or lentivirus and the ITR sequences of AAV (Kootstra et al, Ann. Rev. Pharm. Toxicol., 43:413-439, 2003).

Cells may be cultured in vitro or genetically engineered, for example. Host cells can be obtained from normal or affected subjects, including healthy humans, cancer patients, and patients with an infectious disease, private laboratory deposits, public culture collections such as the American Type Culture Collection, or from commercial suppliers.

Exemplary mammalian host cells include, without limitation, cells derived from humans, monkeys, and rodents (see, for example, Kriegler in “Gene Transfer and Expression: A Laboratory Manual,” 1990, New York, Freeman & Co.). These include monkey kidney cell lines transformed by SV40 (e.g., COS-7, ATCC CRL 1651); human embryonic kidney lines (e.g., 293, 293-EBNA, or 293 cells subcloned for growth in suspension culture, Graham et al, J Gen Virol 1977, 36:59); baby hamster kidney cells (e.g., BHK, ATCC CCL 10); Chinese hamster ovary-cells-DHFR (e.g., CHO, Urlaub and Chasin, Proc Natl Acad Sci USA 1980, 77:4216); mouse sertoli cells (Mather, Biol Reprod 1980, 23:243-251); mouse fibroblast cells (e.g., NIH-3T3), monkey kidney cells (e.g., CV1 ATCC CCL 70); African green monkey kidney cells. (e.g., VERO-76, ATCC CRL-1587); human cervical carcinoma cells (e.g., HELA, ATCC CCL 2); canine kidney cells (e.g., MDCK, ATCC CCL 34); buffalo rat liver cells (e.g., BRL 3A, ATCC CRL 1442); human lung cells (e.g., W138, ATCC CCL 75); human liver cells (e.g., Hep G2, HB 8065); and mouse mammary tumor cells (e.g., MMT 060562, ATCC CCL51). Illustrative cancer cell types for expressing the fusion proteins described herein include mouse fibroblast cell line, NIH3T3, mouse Lewis lung carcinoma cell line, LLC, mouse mastocytoma cell line, P815, mouse lymphoma cell line, EL4 and its ovalbumin transfectant, E.G7, mouse melanoma cell line, B16F10, mouse fibrosarcoma cell line, MC57, human small cell lung carcinoma cell lines, SCLC #2 and SCLC #7, human lung adenocarcinoma cell line, e.g., AD100, and human prostate cancer cell line, e.g., PC-3.

Cells that can be used for production and secretion of gp96-Ig fusion proteins in vivo include, without limitation, epithelial cells, endothelial cells, keratinocytes, fibroblasts, muscle cells, hepatocytes; blood cells such as T lymphocytes, B lymphocytes, monocytes, macrophages, neutrophils, eosinophils, megakaryocytes, or granulocytes, various stem or progenitor cells, such as hematopoietic stem or progenitor cells (e.g., as obtained from bone marrow), umbilical cord blood, peripheral blood, fetal liver, etc., and tumor cells (e.g., human tumor cells). The choice of cell type depends on the type of tumor or infectious disease being treated or prevented, and can be determined by one of skill in the art.

Different host cells have characteristic and specific mechanisms for post-translational processing and modification of proteins. A host cell may be chosen which modifies and processes the expressed gene products in a specific fashion similar to the way the recipient processes its heat shock proteins (hsps). For the purpose of producing large amounts of gp96-Ig, it can be preferable that the type of host cell has been used for expression of heterologous genes, and is reasonably well characterized and developed for large-scale production processes. In some embodiments, the host cells are autologous to the patient to whom the present fusion or recombinant cells secreting the present fusion proteins are subsequently administered.

In some embodiments, an expression construct as provided herein can be introduced into an antigenic cell. As used herein, antigenic cells can include preneoplastic cells that are infected with a cancer-causing infectious agent, such as a virus, but that are not yet neoplastic, or antigenic cells that have been exposed to a mutagen or cancer-causing agent, such as a DNA-damaging agent or radiation, for example. Other cells that can be used are preneoplastic cells that are in transition from a normal to a neoplastic form as characterized by morphology or physiological or biochemical function.

Typically, the cancer cells and preneoplastic cells used in the methods provided herein are of mammalian origin. Mammals contemplated include humans, companion animals (e.g., dogs and cats), livestock animals (e.g., sheep, cattle, goats, pigs and horses), laboratory animals (e.g., mice, rats and rabbits), and captive or free wild animals.

In some embodiments, cancer cells (e.g., human tumor cells) can be used in the methods described herein. In some embodiments, the cell is a human tumor cell. In some embodiments, the cell is an irradiated or live and attenuated human tumor cell. The cancer cells provide antigenic peptides that become associated non-covalently with the expressed gp96-Ig fusion proteins. Cell lines derived from a preneoplastic lesion, cancer tissue, or cancer cells also can be used, provided that the cells of the cell line have at least one or more antigenic determinant in common with the antigens on the target cancer cells. Cancer tissues, cancer cells, cells infected with a cancer-causing agent, other preneoplastic cells, and cell lines of human origin can be used. Cancer cells excised from the patient to whom ultimately the fusion proteins ultimately are to be administered can be particularly useful, although allogeneic cells also can be used. In some embodiments, a cancer cell can be from an established tumor cell line such as, without limitation, an established non-small cell lung carcinoma (NSCLC), melanoma, ovarian cancer, renal cell carcinoma, prostate carcinoma, sarcoma, breast carcinoma, squamous cell carcinoma, head and neck carcinoma, hepatocellular carcinoma, pancreatic carcinoma, or colon carcinoma cell line. In one aspect, the cancer cell is the human lung cancer cell line. In some embodiments, the lung cancer cell line expresses various known lung cancer antigens.

In some embodiments, the present fusion proteins allow for the presentation of various tumor cell antigens. For instance, in some embodiments, the present vaccine protein fusions (e.g., gp96 fusions) chaperone these various tumor antigens. In some embodiments, the tumor cells secrete a variety of antigens. Illustrative, but non-limiting, antigens that can be secreted and/or presented are: Cancer/testis antigen 1A (CTAG1A) and its immunogenic epitopes CT45A6, CT45A3, CT45A1, CT45A5, sperm autoantigenic protein 17 (SPA17), sperm associated antigen 6 (SPAG6), sperm associated antigen 8(SPAG8), ankyrin repeat domain 45 (ANKRD45), lysine demethylase 5B (KDM5B), sperm acrosome associated 3 (SPACA3), sperm flagellar 2 (SPEF2), Hemogen (HEMGN), protease, serine 50 (PRSS50), PDZ binding kinase (PBK), Transketolase-like protein 1 (TKTL1), TGFB induced factor homeobox 2 like, X-linked (TGIF2LX), variable charge, X-linked (VCX), chromosome X open reading frame 67 (CXORF67), MART-1/Melan-A, gp100, Dipeptidyl peptidase IV (DPPIV), adenosine deaminase-binding protein (ADAbp), cyclophilin b, Colorectal associated antigen (CRC)-0017-1A/GA733, Carcinoembryonic Antigen (CEA) and its immunogenic epitopes CAP-1 and CAP-2, etv6, aml1 Prostate Specific Antigen (PSA) and its immunogenic epitopes PSA-1, PSA-2, and PSA-3, prostate-specific membrane antigen (PSMA), T-cell receptor/CD3-zeta chain, MAGE-family of tumor antigens (e.g., MAGE-A1, MAGE-A2, MAGE-A3, MAGE-A4, MAGE-A5, MAGE-A6, MAGE-A7, MAGE-A8, MAGE-A9, MAGE-A10, MAGE-A11, MAGE-A12, MAGE-Xp2 (MAGE-B2), MAGE-Xp3 (MAGE-B3), MAGE-Xp4 (MAGE-B4), MAGE-C1, MAGE-C2, MAGE-C3, MAGE-C4, MAGE-05), GAGE-family of tumor antigens (e.g., GAGE-1, GAGE-2, GAGE-3, GAGE-4, GAGE-5, GAGE-6, GAGE-7, GAGE-8, GAGE-9, GAGE12G, GAGE12F, GAGE12I), BAGE, RAGE, LAGE-1, NAG, GnT-V, MUM-1, CDK4, tyrosinase, p53, MUC family, HER2/neu, p21ras, RCAS1, α-fetoprotein, E-cadherin, α-catenin, β-catenin and γ-catenin, p120ctn, gp100 Pmel117, PRAME, NY-ESO-1, cdc27, adenomatous polyposis coli protein (APC), fodrin, Connexin 37, Ig-idiotype, p15, gp75, GM2 and GD2 gangliosides, viral products such as human papilloma virus proteins, Smad family of tumor antigens, Imp-1, NA, EBV-encoded nuclear antigen (EBNA)-1, brain glycogen phosphorylase, SSX-1, SSX-2 (HOM-MEL-40), SSX-1, SSX-4, SSX-5, SCP-1 CT-7, c-erbB-2, CD19, CD20, CD22, CD30, CD33, CD37, CD56, CD70, CD74, CD138, AGS16, MUC1, GPNMB, Ep-CAM, PD-L1, PD-L2, and PMSA.

PD-1 is a cell surface receptor that is a member of the CD28 family of T-cell regulators, within the immunoglobulin superfamily of receptors. The human PD-1 gene is located at chromosome 2q37, and the full-length PD-1 cDNA encodes a protein with 288 amino acid residues with 60% homology to murine PD-1. It is present on CD4− CD8− (double negative) thymocytes during thymic development and is expressed upon activation in mature hematopoietic cells such as T and B cells, NKT cells and monocytes after prolonged antigen exposure.

The principal method for targeting PD-1 clinically has been through the development of genetically engineered monoclonal antibodies that inhibit either PD-1 or PD-L1 function. PD-L1 has also been shown to suppresses T-cell proliferation and cytokine production; however, the exact pathways in cancer remain unclear. Cancer cells drive high expression levels of PD-L1 on their surface, allowing activation of the inhibitory PD-1 receptor on any T cells that infiltrate the tumor microenvironment, effectively switching those cells off. Indeed, upregulation of PD-L1 expression levels has been demonstrated in many different cancer types (e.g., melanoma [40%-100%], NSCLC [35%-95%], and multiple myeloma [93%]), and high levels of PD-L1 expression have been linked to poor clinical outcomes. Furthermore, tumor-infiltrating T cells (TILs) have been shown to express significantly higher levels of PD-1 than T cells that infiltrate normal tissue. It is thought that the tumor microenvironment may secrete pro-inflammatory cytokines, including interferon-gamma (IFNγ) to upregulate the expression of PD-1 on tumor-infiltrating T cells to ensure that they can respond to the high levels of PD-L1 expressed on the tumor.

In some embodiments, the anti-PD-1 antibody or antigen binding fragment thereof is Nivolumab, Pembrolizumab, Pidilizumab, BMS-936559, Atezolizumab or Avelumab.

In some embodiments, the anti-PD-L1 antibody or antigen binding fragment thereof is durvalumab.

Two anti-PD-1 antibodies of particular interest, nivolumab and pembrolizumab have been approved in the U.S. for a number of different cancers and there are a large number of additional anti-PD-1 antibodies in clinical testing.

Accordingly, in some embodiments, the anti-PD-1 antibody for use in combination with Viagenpumatucel-L is nivolumab. Suitable doses and dosing regimens are described below.

In some embodiments, the anti-PD-1 antibody for use in combination with Viagenpumatucel-L is pembrolizumab. Suitable doses and dosing regimens are described below.

The combinations and methods disclosed herein are suitable for treating cancer or inhibiting cancer cell proliferation, such as lung cancer. In some embodiments, the lung cancer is a Non-small lung cancer, such as squamous cell carcinoma, adenocarcinoma, and large cell lung carcinoma.

In general, the present invention provides for increased efficacy of anti-PD-1 or anti-PD-L1 antibodies in patients who typically do not significantly benefit from anti-PD-1 therapies. For example, patients that have low or negative expression of PD-L1 on their tumors generally do not significantly benefit from anti-PD-1 therapy. However, surprisingly, as shown herein, the combination of Viagenpumatucel-L and anti-PD-1 antibodies is equally efficacious irrespective of the PD-L1 status of the patient's tumor(s). Similarly, patients with low TIL status are generally not very responsive to anti-PD-1 therapy. Again, surprisingly, the combination of Viagenpumatucel-L and anti-PD-1 antibodies is equally efficacious irrespective of the TIL status of the patient's tumor(s).

Thus, the invention provides methods of determining the PD-L1 status of the NSCLC patient. Generally, this is done by obtaining one or more tumor biopsies from the patient and testing for PD-L1 status as is known in the art. This is generally done using immunohistochemical (IHC) assays on biopsied tumor samples using labeled antibodies as is known in the art, and is generally scored as PD-L1^{high} (high levels of staining), PD-L1^{low} (low levels of staining) and PD-L1^{negative} (<1%, no staining detected). In some embodiments, an anti-PD-L1 staining is used with a standardized immunohistochemical assay, PD-L1^{high} corresponds to ≥50% PD-L1 tumor type cells that stain positive, PD-L1^{low} is 49-1% PD-L1 tumor type cells that stain positive and in PD-L1^{negative} <1% staining detected. FACS statining of disassociating tumor biopsies using anti-PD-L1 antibodies may also be conducted.

As is known in the art, patients generally do better with anti-PD-1 antibody treatment if they are PD-L1^{high}. However, the present invention enables the use of anti-PD-1 antibodies in combination with Viagenpumatucel-L even if patients are PD-L1^{low} and PD-L1^{negative} to produce synergist effects.

Additionally, in some embodiments, the TIL status of a patient can be determined. As outlined herein, tumors that have low amounts of CD8+ TILs in the tumor microenvironment are generally considered “cold” tumors, e.g. TIL^{low}, which are less likely to respond to immune-oncology treatments than tumors with high amounts of CD8+ TILs (TIL^{high}).

As above, TIL status is generally assessed as is known in the art, e.g. by disassociating tumor biopsies and using FACS sorting for CD8+ cells as is known in the art or by conducting immunohistochemistry (IHC) staining of tumor biopsy samples. In some embodiments, anti-CD8 antibody staining is used to evaluate the percentage of CD8+ cells in the tumor stroma. TIL^{high} corresponds to >about 10% CD8⁺ cells in the tumor biopsy and TIL^{low} corresponds to <about 10% CD8⁺ cells in the tumor sample.

Thus, while patients generally do better with anti-PD-1 antibody protocols when their TIL status is TIL^{high}, the present invention enables the use of anti-PD-1 antibodies in combination with Viagenpumatucel-L even if patients are TIL^{low} to produce synergist effects.

The invention provides the co-administration of Viagenpumatucel-L and anti-PD-1 antibodies to patients suffering from NSCLC.

In embodiments, the patient has experienced disease progression after receiving a therapy. In embodiments, the therapy is an immune checkpoint inhibitor therapy. In embodiments, the therapy comprises chemotherapy. In embodiments, the patient is a poor responder to the immune checkpoint inhibitor therapy. In embodiments, the patient has failed the immune checkpoint inhibitor therapy. In embodiments, the disease in the patient has progressed even when administered the immune checkpoint inhibitor therapy. In some embodiments, the patient previously received CPI therapy, however, disease progressed after 6 months or longer of treatment.

In some embodiments, the methods of the present disclosure involve administering a cell comprising a vector encoding a modified and secretable heat shock protein (i.e., gp96-Ig) in combination with an anti-PD-1 antibody. In some embodiments, the number of cells administered range from about 100,000 cells to about 20 million cells.

In some embodiments, a low dose amount of the cell is administered to a subject. For example, the number of cells administered to the subject can be about 100,000 cells, about 150,000 cells, about 200,000 cells, about 250,000 cells, about 300,000 cells, about 350,000 cells, about 400,000 cells, about 450,000 cells, about 500,000 cells, about 550,000 cells, about 600,000 cells, about 650,000 cells, about 700,000 cells, about 750,000 cells, about 800,000 cells, about 850,000 cells, about 900,000 cells, about 950,000 cells, or about 1 million cells. In an embodiment, about 1 million cells are administered to a subject.

In some embodiments, a high dose amount of the cell is administered to a subject. For example, the number of cells administered to the subject can be about 2 million cells, about 3 million cells, about 4 million cells, about 5 million cells, about 6 million cells, about 7 million cells, about 8 million cells, about 9 million cells, about 10 million cells, about 11 million cells, about 12 million cells, about 13 million cells, about 14 million cells, about 15 million cells, about 16 million cells, about 17 million cells, about 18 million cells, about 19 million cells, or about 20 million cells.

In the combination therapy regimens outlined herein, a dose that finds particular use is 1×10⁷ Viagenpumatucel-L cells, given as an injection.

In many embodiments, the Viagenpumatucel-L cells are given weekly for a period of at least about 6 weeks to at least about 16 weeks in combination with every other week (biweekly) IV infusions of anti-PD-1 antibodies such as nivolumab.

The anti-PD-1 antibodies are administered as is known in the art for appropriate dosing of NSCLC patients. In general, a dose of 240 mg is administered biweekly.

In some embodiments, the Viagenpumatucel-L cells, e.g. at a dose of about 1×10⁷ cells, is given in combination with a regimen of anti-PD-1 or anti-PD-L1 antibody treatment. For instance, in some embodiments, the Viagenpumatucel-L cells, e.g. at a dose of about 1×10⁷ cells, are combined with a single dose regimen of Nivolumab (3 mg/kg intravenously every two weeks). In some embodiments, the Viagenpumatucel-L cells, e.g. at a dose of about 1×10⁷ cells, are combined with a 2 week dosing schedule of Nivolumab, e.g. 240 mg, optionally for a total of about 6 weeks, or optionally for a total of about 16 weeks. In some embodiments, the Viagenpumatucel-L cells, e.g. at a dose of about 1×10⁷ cells, are combined with a 4-week dosing schedule of Nivolumab, e.g. 480 mg, optionally infused every 30 minutes every 4 weeks.

In some embodiments, useful dosing regimens of the two components are as follows:

REGIMEN 1

| | Viagenpumatucel-L | Nivolumab |
|---|---|---|
| Week | dose | dose |
| 1 | 1 × 10⁷ cells | 240 mg |
| 2 | 1 × 10⁷ cells | none |
| 3 | 1 × 10⁷ cells | 240 mg |
| 4 | 1 × 10⁷ cells | none |
| 5 | 1 × 10⁷ cells | 240 mg |
| 6 | 1 × 10⁷ cells | none |
| 7 | 1 × 10⁷ cells | 240 mg |
| 8 | 1 × 10⁷ cells | none |
| 9 | 1 × 10⁷ cells | 240 mg |
| 10 | 1 × 10⁷ cells | none |
| 11 | 1 × 10⁷ cells | 240 mg |
| 12 | 1 × 10⁷ cells | none |
| 13 | 1 × 10⁷ cells | 240 mg |
| 14 | 1 × 10⁷ cells | none |
| 15 | 1 × 10⁷ cells | 240 mg |
| 16 | 1 × 10⁷ cells | none |

REGIMEN 2

| | Viagenpumatucel-L | Nivolumab |
|---|---|---|
| Week | dose | dose |
| 1 | 1 × 10⁷ cells | 240 mg |
| 2 | 1 × 10⁷ cells | none |
| 3 | 1 × 10⁷ cells | 240 mg |
| 4 | 1 × 10⁷ cells | none |
| 5 | 1 × 10⁷ cells | 240 mg |
| 6 | 1 × 10⁷ cells | none |

As will be appreciated by those in the art, patients can be kept on these protocols week by week until disease progression and/or unacceptable toxicities or adverse events.

In one aspect, the methods of the present disclosure provide a combination comprising a low dose amount of a cell line that expresses a modified and secretable heat shock protein (i.e., gp96-Ig) and an immune checkpoint inhibitor, and a method of using the combination to treat diseases, such as those the cause of which can be influenced by modulating immune cell profiling of Tumor infiltrating lymphocytes (TIL) and/or other proteins, e.g., cancer. In some embodiments, the present disclosure features a combination comprising a low dose amount of a cell line that expresses a modified and secretable heat shock protein (i.e., gp96-Ig) and an anti-PD-1 antibody or antigen binding fragment thereof (e.g., Nivolumab).

The method comprises administering to a subject in need thereof an effective amount of a low dose amount of a cell line that expresses a modified and secretable heat shock protein (i.e., gp96-Ig) and an anti-PD-1 antibody or antigen binding fragment thereof of (e.g., Nivolumab)., e.g., by inhibiting tumor growth, reducing and intra-tumor T regulatory cell population and/or increasing CD8/T-regulatory cell ratio in tumors.

The present disclosure further provides uses of any methods or combinations described herein in the manufacture of medicament for treating a disease. Such diseases include, for example, cancer, a precancerous condition, or a disease influenced by modulating the immune cell profiling of Tumor infiltrating lymphocytes (TIL) and/or other proteins.

In some embodiments, the present disclosure provides a combination therapy involving a cell line that contains a vector encoding a modified and secretable heat shock protein (i.e., gp96-Ig). Administration of a low dose amount of the cell line in combination with an immune checkpoint inhibitor, such as an anti-PD-1 monoclonal antibody or antigen binding fragment thereof (e.g., Nivolumab) reduces NSCLC recurrence.

The method comprises administering to a subject in need thereof an effective amount of a low dose amount of a cell line that expresses a modified and secretable heat shock protein (i.e., gp96-Ig) and an anti-PD-1 antibody (e.g., Nivolumab), by inhibiting tumor growth, reducing and intra-tumor T regulatory cell population and/or increasing CD8/T-regulatory cell ratio in tumors.

The combinations and methods disclosed herein are suitable for treating cancer or inhibiting cancer cell proliferation, such as squamous cell carcinoma, adenocarcinoma, and large cell carcinoma, e.g., Non-small lung cancer.

In some embodiments, the methods provided herein can be useful for stimulating an immune response against a tumor (e.g., lung tumor). In some embodiments, such immune response is useful in treating or alleviating a sign or symptom associated with the tumor. As used herein, by “treating” is meant reducing, preventing, and/or reversing the symptoms in the individual to which a vector as described herein has been administered, as compared to the symptoms of an individual not being treated. A practitioner will appreciate that the methods described herein are to be used in concomitance with continuous clinical evaluations by a skilled practitioner (physician or veterinarian) to determine subsequent therapy. Such evaluations will aid and inform in evaluating whether to increase, reduce, or continue a particular treatment dose, mode of administration, etc.

In some embodiments, the methods of the invention can increase the activation or proliferation of tumor antigen specific T cells in a subject. For example, the activation or proliferation of tumor antigen specific T cells in the subject can be increased by at least 5% (e.g., including for example at least about 10%, 20%, 30%, 40%, 60%, 70%, 80%, 90%, or 100%) as compared to the level of activation or proliferation of tumor antigen specific T cells in the subject prior to the administration. In an embodiment, the increase is compared to administration of the immune checkpoint inhibitor (e.g., anti-PD-1 antibody) alone.

In some embodiments, the present methods can increase the activation or proliferation of CD8+ T cells in a subject. For example, the activation or proliferation of CD8+ T cells in the subject can be increased by at least 5% (e.g., including for example at least about 10%, 20%, 30%, 40%, 60%, 70%, 80%, 90%, or 100%) as compared to the level of activation or proliferation of CD8+ T cells in the subject prior to the administration. In some embodiments, the CD8+ T cell is an IFN-γ secreting T cell. In an embodiment, the increase is compared to administration of the immune checkpoint inhibitor alone. In some embodiments, the activation or proliferation of CD8+ T cells (e.g., CD8+ T cell that secrete IFN-γ) can be assessed by Enzyme-Linked ImmunoSpot (ELISPOT) assays performed, for example, on peripheral blood lymphocytes derived from the subject.

In some embodiments, the methods of the invention effectively induce and/or activate tumor infiltrating lymphocytes (TILs) and/or increase the number of such TILs in the subject. For example, the induction and/or activation and/or increase in the number of such TILs in the subject can be by at least 5% (e.g., including for example at least about 10%, 20%, 30%, 40%, 60%, 70%, 80%, 90%, or 100%) as compared to prior to administration. In an embodiment, the increase is compared to administration of the immune checkpoint inhibitor (e.g., anti-PD-1 antibody) alone.

In some embodiments, the methods of the invention effectively reduce the recurrence rate of lung cancer in a subject. In some embodiments, the methods of the disclosure effectively reduce the recurrence rate of lung cancer in a subject who has been treated with a combination of a low dose amount of a cell line that expresses a modified and secretable heat shock protein (i.e., gp96-Ig) and an immune checkpoint inhibitor such as, anti-PD-1 antibody. In some embodiments, the anti-PD-1 antibody or antigen binding fragment thereof is Nivolumab. In some embodiments, the checkpoint inhibitor includes durvalumab, ipilimumab, pembrolizumab, pidilizumab, BMS-936559, atezolizumab or avelumab.

In an embodiment, the methods of the disclosure are more effective in treating (e.g., in reducing cancer recurrence) those subjects that has been treated with a combination of a low dose amount of the cell line of the disclosure and an immune checkpoint inhibitor such as, anti-PD-1 antibody than subjects who have been treated with a combination of a high dose amount of the cell line of the disclosure and an immune checkpoint inhibitor such as, anti-PD-1 antibody.

In one aspect, the methods of the disclosure are more effective in treating (e.g., in reducing cancer recurrence) those subjects that has been treated with a combination of a low dose amount of the cell line of the invention and an immune checkpoint inhibitor such as, anti-PD-1 antibody or antigen binding fragment thereof, than subjects who have been treated with the immune checkpoint inhibitor such as, anti-PD-1 antibody or antigen binding fragment thereof alone.

In an embodiment, the administration of a combination of a cell line of the invention and the immune checkpoint inhibitor such as, anti-PD-1 antibody or antigen binding fragment thereof induces a robust increase in immune response following treatment. In an embodiment, the robust increase in immune response is defined as an increase of at least 2 fold above the baseline in the activation or proliferation of CD8+ T cells (e.g., CD8+ T cell that secrete IFN-γ) as measured, for example, by ELISPOT. In an embodiment, the methods of the invention are more effective in treating a subject who exhibits a robust immune response following treatment than a subject who does not exhibit such an immune response. In such an embodiment, the subject may be treated with a combination of a low dose amount of the cell line of the invention and an immune checkpoint inhibitor, (e.g., anti-PD-1 antibody).

In some embodiments, the present disclosure provides a method for treating a subject who shows a high number of tumor infiltrating lymphocytes (TILs) prior to treatment by administering to the subject a combination of a cell line of the disclosure and an immune checkpoint inhibitor, (e.g., anti-PD-1 antibody). In some embodiments, a high number of TILs refers to a TIL number of higher than 10% of the cells in the tumor microenvironment.

In other embodiments, the present invention provides a method for treating a subject who shows a low number of tumor infiltrating lymphocytes (TILs) prior to treatment by administering to the subject a combination of a cell line of the disclosure and an immune checkpoint inhibitor, (e.g., anti-PD-1 antibody). In some embodiments, a low number of TILs refers to a TIL number of less than or equal to 10% of the cells in the tumor microenvironment. In some embodiments, the methods of the disclosure may be more effective in treating those subjects with a low number of tumor infiltrating lymphocytes (TILs) than subjects with a high number of TILs. In some embodiments, the present disclosure provides methods of treating subjects with a low number of TILs with a combination of a cell line of the disclosure and an immune checkpoint inhibitor, (e.g., anti-PD-1 antibody).

As used herein, the terms “effective amount” and “therapeutically effective amount” refer to an amount sufficient to provide the desired therapeutic (e.g., anti-cancer or anti-tumor) effect in a subject (e.g., a human diagnosed as having cancer). Anti-tumor and anti-cancer effects include, without limitation, modulation of tumor growth (e.g., tumor growth delay), tumor size, or metastasis, the reduction of toxicity and side effects associated with a particular anti-cancer agent, the amelioration or minimization of the clinical impairment or symptoms of cancer, extending the survival of the subject beyond that which would otherwise be expected in the absence of such treatment, and the prevention of tumor growth in an animal lacking tumor formation prior to administration, i.e., prophylactic administration. In an embodiment, the present invention reduces or prevents cancer recurrence (e.g., lung cancer recurrence).

The methods described herein are useful for various aspects of lung cancer treatment. In some embodiments, there is provided a method of inhibiting lung cancer cell proliferation (such as lung cancer tumor growth) in an individual. In some embodiments, at least about 10% (including for example at least about 20%, 30%, 40%, 60%, 70%, 80%, 90%, or 100%) cell proliferation is inhibited. In some embodiments, less than about 20% of cell proliferation is inhibited.

In some embodiments, there is provided a method of inhibiting lung cancer tumor metastasis in an individual. In some embodiments, at least about 10% (including for example at least about any of 20%, 30%, 40%, 60%, 70%, 80%, 90%, or 100%) metastasis is inhibited.

In some embodiments, there is provided a method of reducing the incidence or burden of pre-existing lung cancer tumor metastasis (such as pulmonary metastasis or metastasis to the lymph node) in an individual. In some embodiments, at least about 10% (including for example at least about 20%, 30%, 40%, 60%, 70%, 80%, 90%, or 100%) metastasis is reduced.

In some embodiments, there is provided a method of reducing lung cancer tumor size in an individual. In some embodiments, the tumor size is reduced at least about 10% (including for example at least about 20%, 30%, 40%, 60%, 70%, 80%, 90%, or 100%).

In some embodiments, there is provided a method of reducing lung cancer recurrence in an individual. In some embodiments, the recurrence of lung cancer is reduced by at least about 10% (including for example at least about 20%, 30%, 40%, 60%, 70%, 80%, 90%, or 100%).

In some embodiments, there is provided a method of prolonging time to disease progression of lung cancer in an individual. In some embodiments, the method prolongs the time to disease progression by at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51 or 52 weeks. In some embodiments, the method prolongs the time to disease progression by at least 1, 2, 3, 4, 5, 6, 7, 8, 9, or 10 years.

In some embodiments, there is provided a method of prolonging survival of an individual having lung cancer. In some embodiments, the method prolongs the survival of the individual by at least 1, 2, 3, 4, 5, 6, 7, 8, 9, 10, 11, 12, 18, 24 months. In some embodiments, the method prolongs the survival of the individual by at least about 1, 2, 3, 4, 5, 6, 7, 8, 9, or 10 years.

In some embodiments, there is provided a method of alleviating one or more symptoms in an individual having lung cancer, (e.g., NSCLC)

### EXAMPLES

In order that the invention disclosed herein may be more efficiently understood, examples are provided below. It should be understood that these examples are for illustrative purposes only and are not to be construed as limiting the invention in any manner.

### Example 1: A Phase 1b/2 Study of Viagenpumatucel-L (HS-110) in Combination with Multiple Treatment Regimens in Patients with Non-Small Cell Lung Cancer (the “DURGA” Trial)

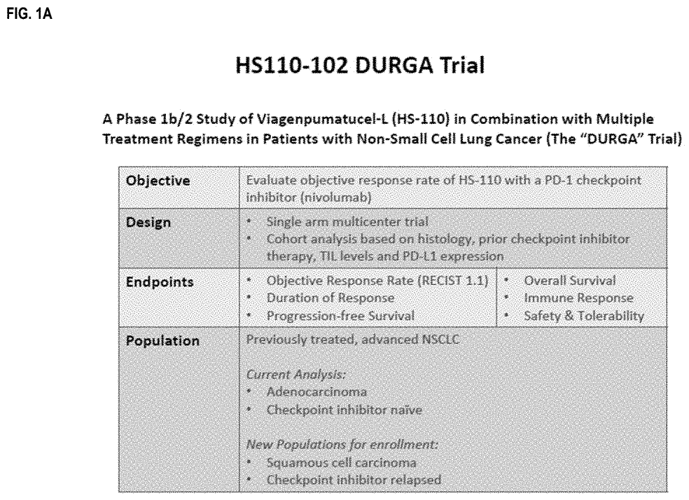

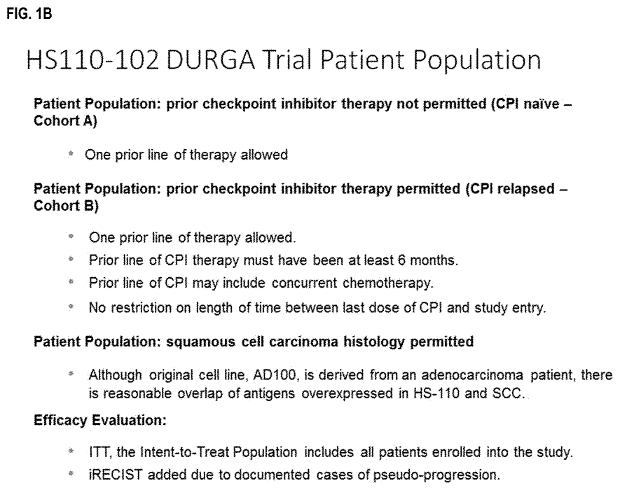

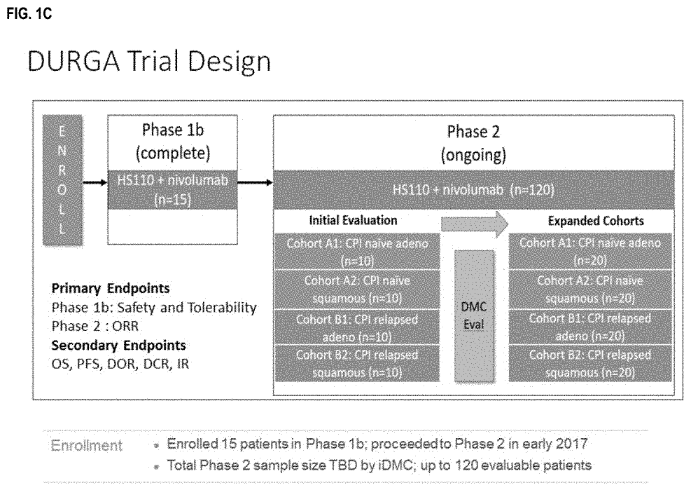

For trial design see FIG. 1A, FIG. 1B, and FIG. 1C.

A goal of the experiments disclosed herein was, inter alia, to evaluate whether vaccination with viagenpumatucel-L combined with strategies to modulate the immune response is safe for patients with non-small cell lung adenocarcinoma who have failed at least one prior line of therapy for incurable or metastatic disease. Response rate of Viagenpumatucel-L (HS-110) with a PD-1 checkpoint inhibitor and second line therapy or greater was evaluated. The patient population included Phase 1b expanded to Phase 2 based upon safety and efficacy.

Patients with NSCLC received 1×10⁷ HS-110 cells weekly for the first 18 weeks, and nivolumab 3 mg/kg or 240 mg every 2 weeks until intolerable toxicity or tumor progression. Tissue was tested at baseline for PD-L1 expression (≥1% high or <1%; negative) and tumor infiltrating lymphocytes (TILs). TIL high was defined by more than 10% CD8+ lymphocytes in the tumor stroma. Two cohorts were studied: Cohort A: patients who had never received checkpoint inhibitor therapy (i.e., checkpoint inhibitor therapy naïve) and Cohort B: patients had previously received checkpoint inhibitor therapy and whose disease progressed after 6 months or longer of treatment.

Tissue from patients in Cohort A was tested at baseline for PD-L1 expression (≥1% or <1%) and tumor infiltrating lymphocytes (TILs). TIL high was defined by more than 10% CD8+ lymphocytes in the tumor stroma. Patients in Cohort A had only one prior treatment, which was chemotherapy. Without limitation, the objectives were safety and objective response rates (ORR), PFS and OS.

### Viagenpumatucel-L+Nivolumab (Low TIL)

Patients with low TIL (tumor-infiltrating lymphocytes) received a combination weekly of viagenpumatucel-L (HS-110) given as injections of 1×10⁷ cells and nivolumab (OPDIVO) for 18 weeks or until treatment discontinuation. 9 patients were initially enrolled (Phase 1b) with an option to expand to 20 patients based on preliminary efficacy (Phase 2). Vaccine was derived from irradiated human lung cancer cells genetically engineered to continually secrete gp96-Ig. Patients received nivolumab per the package insert for the treatment of NSCLC (3 mg/kg as an i.v. infusion over 60 minutes every two weeks) until disease progression or unacceptable toxicity.

### Viagenpumatucel-L+Nivolumab (High TIL)

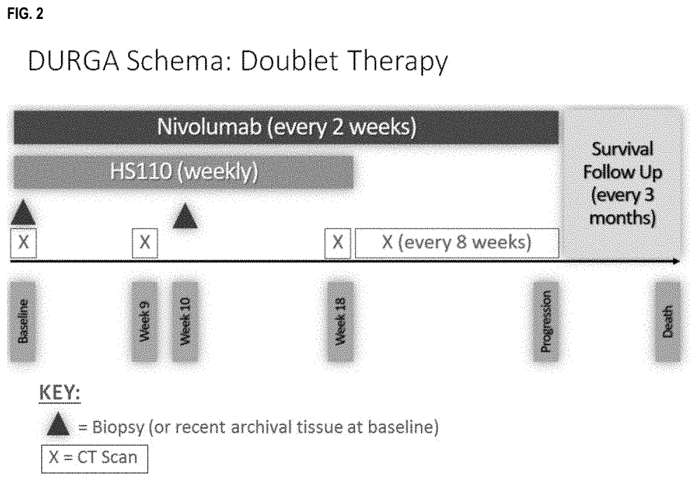

Patients with high TIL (tumor-infiltrating lymphocytes) received a combination of weekly viagenpumatucel-L (HS-110) given as injections of 1×10⁷ cells and Nivolumab (Opdivo) for 18 weeks or until treatment discontinuation. 9 patients were initially enrolled (Phase 1b) with an option to expand to 20 patients based on preliminary efficacy (Phase 2). Vaccine was derived from irradiated human lung cancer cells genetically engineered to continually secrete gp96-lg. Patients received nivolumab per the package insert for the treatment of NSCLC (3 mg/kg as an i.v. infusion over 60 minutes every two weeks) until disease progression or unacceptable toxicity (see FIG. 2).

### Viagenpumatucel-L+Nivolumab (Rollover)

Patients received a combination of weekly viagenpumatucel-L (HS-110) given as injections of 1×10⁷ cells and Nivolumab (Opdivo) for 18 weeks or until treatment discontinuation. This arm allowed patients who have consented but could not be assigned to the high or low TIL groups to enroll and there was no formal limit. Vaccine derived from irradiated human lung cancer cells genetically engineered to continually secrete gp96-Ig was used. Patients received nivolumab per the package insert for the treatment of NSCLC (3 mg/kg as an i.v. infusion over 60 minutes every two weeks) until disease progression or unacceptable toxicity (see FIG. 2).

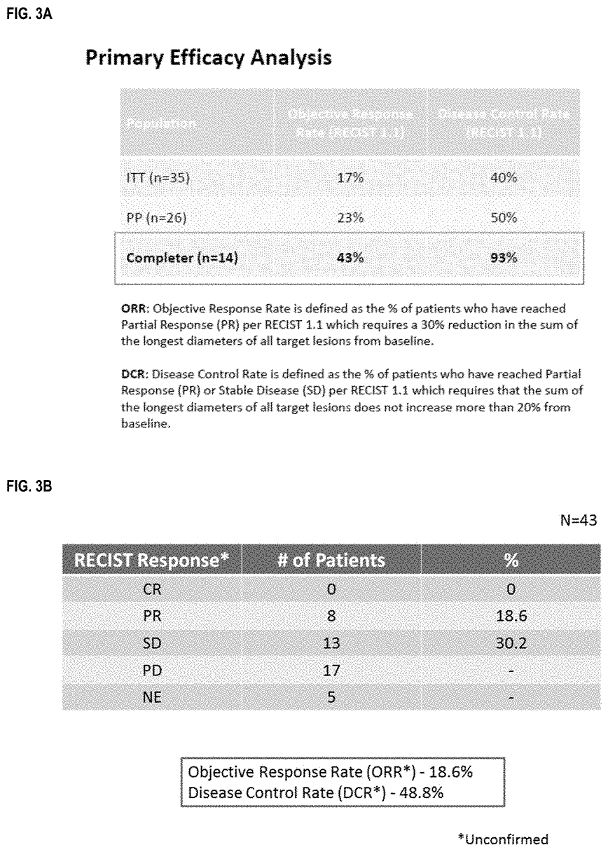

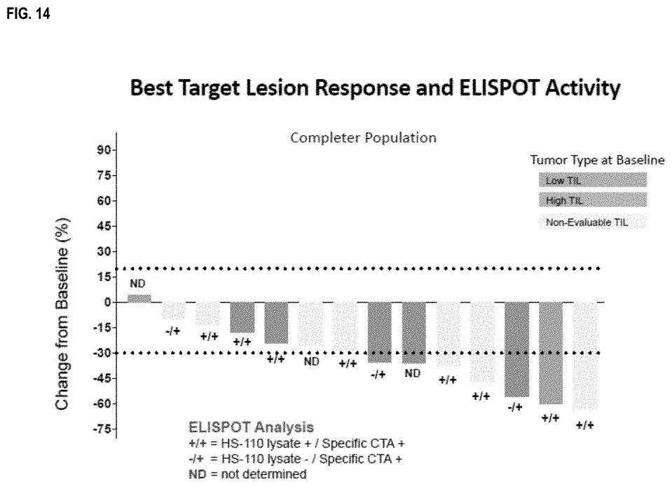

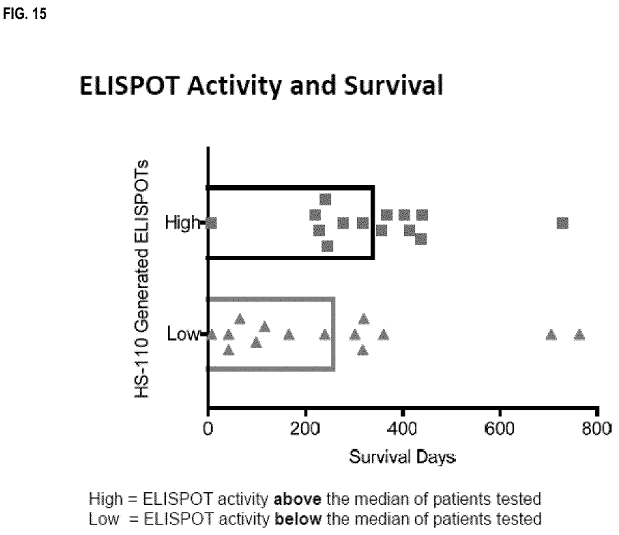

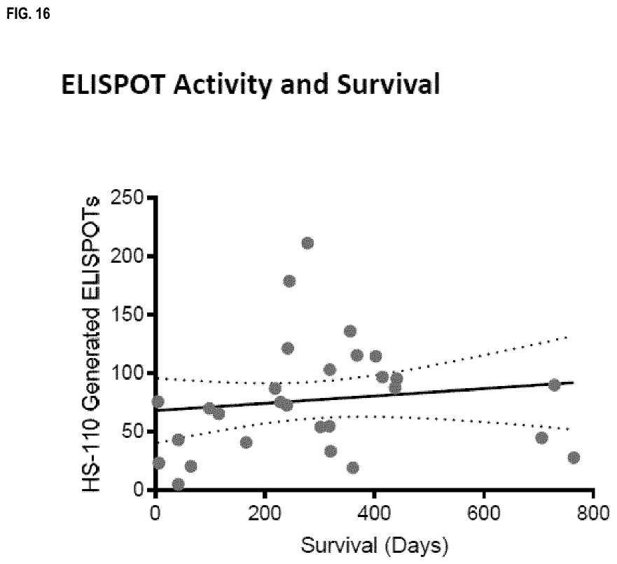

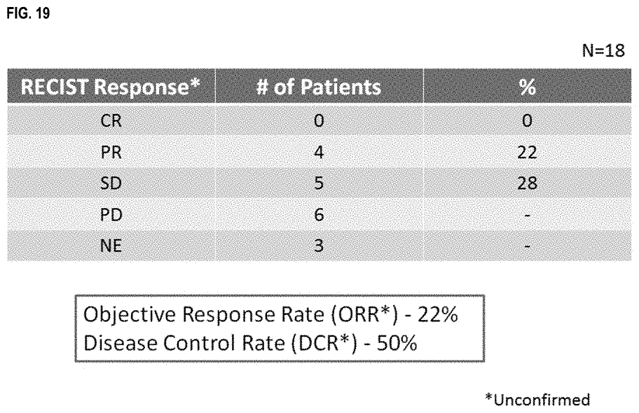

Primary outcome results in Phase 1 b measured safety and tolerability by physical and laboratory examinations up to 3 years and evaluated the safety of each viagenpumatucel-L combination regimen. Primary outcome results in Phase 2 measured objective response Rate (ORR) up to 3 years and evaluated the objective response rate (ORR) by response evaluation criteria in solid tumors (RECIST), see FIG. 3A and FIG. 3B, and see FIG. 19 for CPI progressor results. Secondary outcome results in Phase 1b and measured Objective Response Rate (ORR) up to 3 years and evaluated the Objective Response Rate (ORR) by response evaluation criteria in solid tumors (RECIST). Secondary outcome results in Phase 2 and measured safety and tolerability by physical and laboratory examinations up to 3 years and evaluated the safety of each viagenpumatucel-L combination regimen. The immune response by ELISPOT using a HS-110 lysate was evaluated up to 3 years from peripheral blood following vaccination is characterized (FIG. 14, FIG. 15, FIG. 16) and Overall Survival (OS) and Progression-Free Survival (PFS) is assessed up to 3 years (FIG. 3A).

Additionally, other outcomes such as characterization of T-cell receptor (TCR) repertoire, Peripheral Blood Immune Response by Flow Cytometry Analysis, Total Peripheral Blood Mononuclear Cell (PBMC) counts by Flow Cytometry including Lymphocyte Subsets, Disease Control Rate (DCR) including complete response, partial response or stable response are evaluated up to 3 years. Tumor antigen expression by immunohistochemistry (IHC) and presence of tumor-infiltrating lymphocytes (TILs) in biopsies or archival tissue is assessed during pre-treatment and Tumor-infiltrating lymphocytes and expression of Immunosuppressive Molecules by IHC in biopsies is evaluated nine (9) weeks after first dose of study drug. The proportion of patients who are alive at 6 months following enrollment and 12 months following enrollment is evaluated.

### Clinical Patient Population Criteria

Persons 18 years and older and all sexes were eligible for the study except healthy volunteers. Inclusion criteria: Non-small cell lung adenocarcinoma; one site of measureable disease by RECIST 1.1; Patient populations who have received at least one prior line of therapy for incurable or metastatic NSCLC; life expectancy ≥18 weeks; disease progression at study entry; Eastern Cooperative Oncology Group (ECOG) performance status (PS) ≤1. PS=2 patients may be considered, Central nervous system (CNS) metastases may be permitted but must be treated and neurologically stable; adequate laboratory parameters; willing and able to comply with the protocol and sign informed consent; Female patients who are of childbearing potential and fertile male patients must agree to use an effective form of contraception throughout study participation; willing to provide archival or fresh tumor biopsy at screening and week 10 and suitable for treatment with nivolumab per package insert.

Exclusion Criteria: received systemic anticancer therapy within the previous 21 days; Human immunodeficiency virus (HIV); hepatitis B or C, or severe/uncontrolled infections or concurrent illness, unrelated to the tumor, requiring active therapy; any condition requiring concurrent systemic immunosuppressive therapy; known immunodeficiency disorders, either primary or acquired; known leptomeningeal disease; active malignancies within 12 months with the exception of those with a negligible risk of metastasis or death treated with expected curative outcome; pregnant or breastfeeding; prior treatment with a cancer vaccine for this indication; prior participation in a clinical study of viagenpumatucel-L; administration of a live, attenuated vaccine within 30 days prior to first dose of study drug; active, known or suspected autoimmune disease and prior treatment of the immune checkpoint inhibitor.

The data suggests that the present gp96-based vaccine expands the percentage of patients responding to checkpoint inhibitors by, without wishing to be bound by theory, increasing T cell activity within the tumor, thereby converting “cold” tumors into “hot” tumors (FIG. 3A, FIG. 3B).

Patients with increased levels of tumor infiltrating lymphocytes (TIL) at 10 weeks saw a durable benefit, with 75% (6 out of 8 of these patients) alive at the one-year follow-up point. Additionally, 60% of the patients (3 of the 5 patients) exhibiting low TIL experienced significant tumor reduction, which compares favorably to the 10% response rate of low TIL patients reported for existing data on nivolumab alone.

A strong correlation between T cell activation, tumor reductions and increased overall survival in the 12 of the 15 patients that were evaluable for ELISPOT analysis was observed. Importantly, the timing of immune responses to HS-110 corresponded to the timing of observed clinical responses, and those responses appear to be sustained.

### Example 2: Patient Analysis

A total of 43 patients were enrolled into Cohort A. This patient group consisted of 40 human lung adenocarcinoma (AD) patients and 3 squamous cell carcinoma (SCC) patients. A total of 18 patients were enrolled into cohort B (15 AD and 3 SCC).

### Completer Population Analysis

Viagenpumatucel-L (HS-110; ImPACT) is an allogeneic cellular vaccine derived from a human adenocarcinoma (Ad) cell line transfected with the gp96-Ig fusion protein for the secretion of gp96-cell derived cancer testis antigen (CTA) complexes to drive an adaptive immune response with clinical benefit. Studies have shown that with HS-110 and related gp96-Ig/CTA generated vaccines have shown a correlation between increases in CD8⁺ tumor infiltrating lymphocytes (TIL) and tumor response. As shown in FIGS. 1 and 2, the DURGA trial was designed to evaluate if the combination of HS-110 and nivolumab can generate an adaptive CD8 response with long lasting memory capable of affecting clinical outcomes in NSCLC patients.

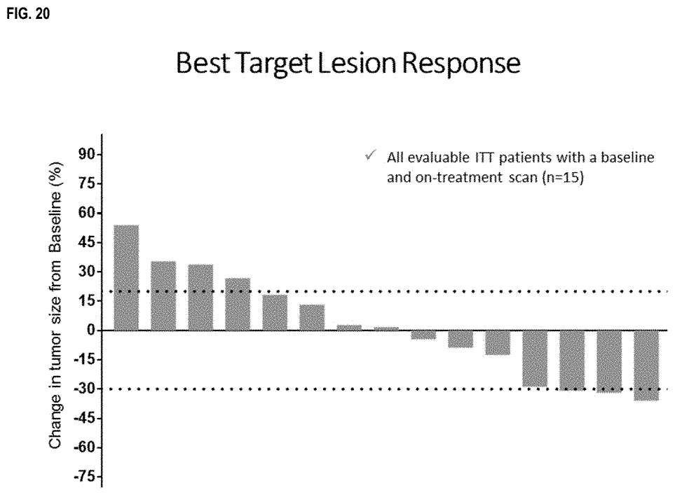

To examine the correlation of adaptive immune response with clinical response after treatment with Viagenpumatucel-L and Nivolumab, patients with advanced and previously treated lung adenocarnoma were treated with weekly doses of HS-110 for 18 weeks and nivolumab 3 mg/kg every 2 weeks until disease progression or death. Biopsy tissue at baseline and at week 10 were tested for levels of CD8⁺ TILs and PD-L1 expression on tumor cells (FIG. 2). Among the 35 patients enrolled, 6 (17%) achieved partial response, 14 (40%) had disease control. Completer analysis demonstrated an ORR and DCR of 43% and 93%, respectively (see FIG. 14). CPI progressor analysis demonstrated an ORR and DCR of 22% and 50%, respectively (see FIG. 20), which demonstrates the treatment with Viagenpumatucel-L and Nivolumab surprisingly restores activity in patients not expected to respond. Thus, the combination of HS-110 and nivolumab was well tolerated, with no additional toxicities compared to single agent checkpoint inhibitors. Positive adaptive immune responses (defined as at least a two-fold increase over nadir) occurred in 86% of patients tested (18 of 21).

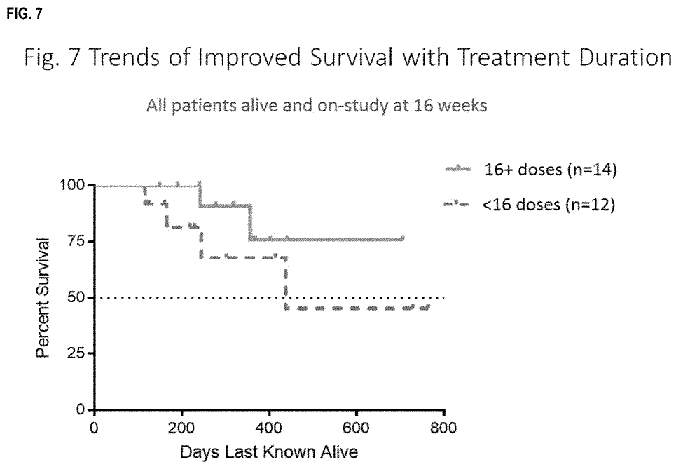

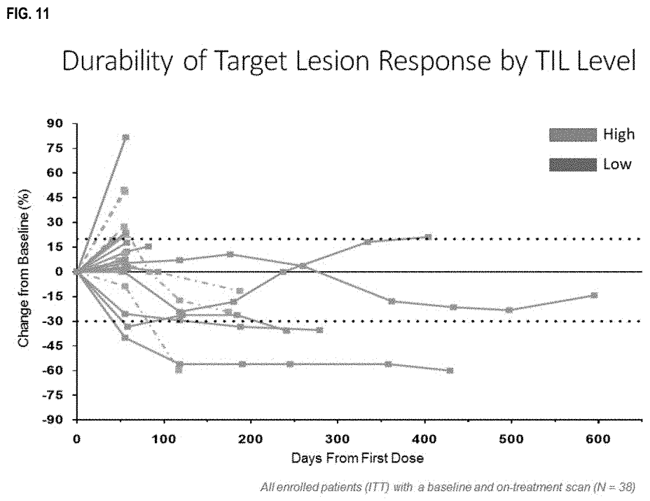

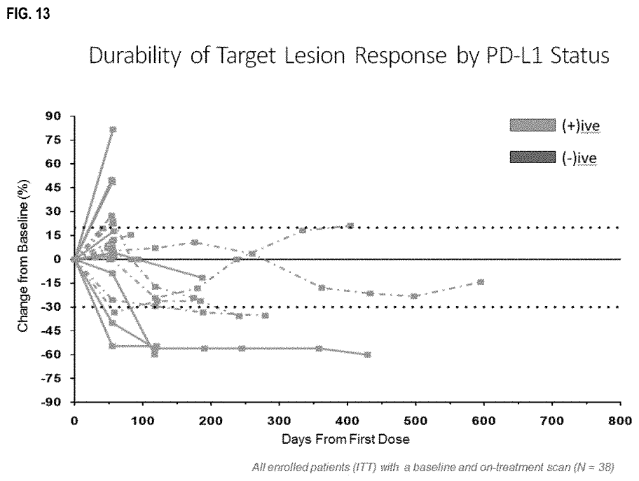

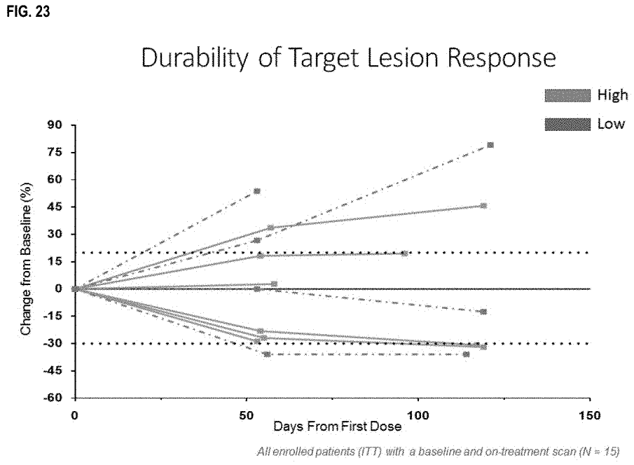

A Time-On-Therapy which demonstrates greater overall survival when comparing the completer population (patients completing study treatment with viagenpumatucel-L, 18 (+/−2) doses) with the non-completer population (patients not completing study treatment with viagenpumatucel-L, <16 doses) showed an increase in Overall Survival (see FIG. 7) and the durability of target lesion response is shown in FIG. 11 (also, see FIG. 5, FIG. 11, FIG. 13, and FIG. 23). Additionally, Low to High TIL were shown to be associated with clinical response. Specifically, the level of CD8⁺ T-cells was dramatically increased after the introduction of combination treatment with viagenpumatucel-L and nivolumab, and is associated with clinical responses of PR per RECIST 1.1.

Peripheral blood was analyzed for immunologic response using the Enzyme-Linked ImmunoSpot (ELISPOT) assay at weeks 1, 4, 7, 13 and at the end of HS-110 treatment. ELISPOT spots generated from stimulating patient PBMCs with whole cell HS-110 vaccine lysates correlates (p=0.06) significantly with the overall survival of patients on therapy, (see FIG. 16).

Intention to Treat and Per Protocol Population analysis

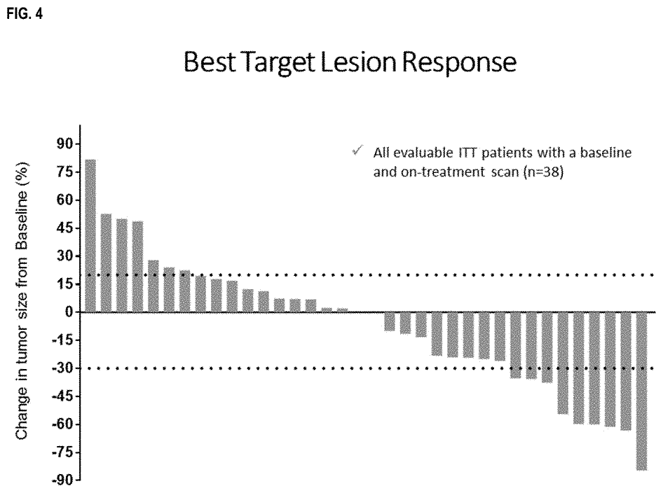

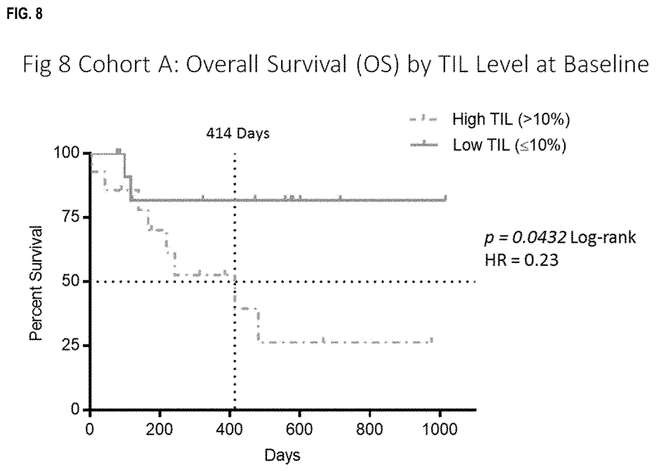

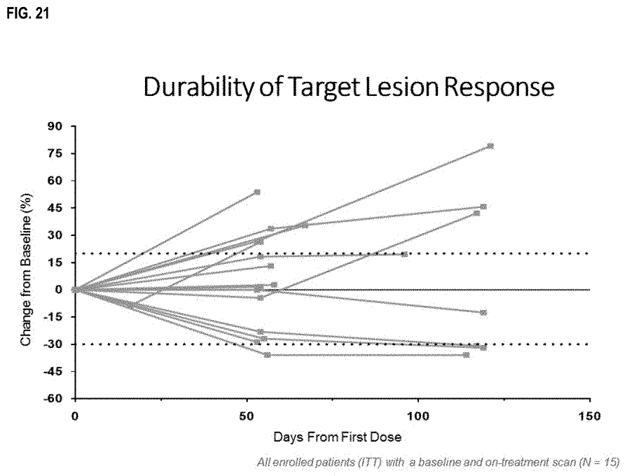

To examine the correlation of adaptive immune response with clinical response after treatment with Viagenpumatucel-L and Nivolumab, patients with advanced and previously treated lung adenocarnoma received doses of HS-110 (weekly) and ≥3 doses nivolumab (anti-PD-1), biweekly. FIG. 4 shows the best target lesion response by RECIST 1.1 in the ITT patient population that was checkpoint inhibitor (CPI) naïve, and FIG. 5 shows the durability of the target lesion response in the CPI naïve per protocol population. Similarly, FIG. 20 shows the best target lesion response by RECIST 1.1 and FIG. 21 is a line graph showing the durability of target lesion response for the CPI progressor ITT patient population. FIG. 8 is a survival plot showing an overall percent survival by tumor infiltrating lymphocyte (TIL) level at baseline for high TIL (>10%; n=14) patients and low TIL (10%; n=14) patients for the CPI naïve population. In FIG. 8, “HR” refers to the hazard ratio, where 1.0 indicates a 100% chance of dying compared to the other group (i.e., low TIL group compared to high TIL group). The results in FIG. 8 demonstrate that there is a 23% chance of dying for the low TIL patient group compared to the high TIL patient group with a significant p value of 0.043.

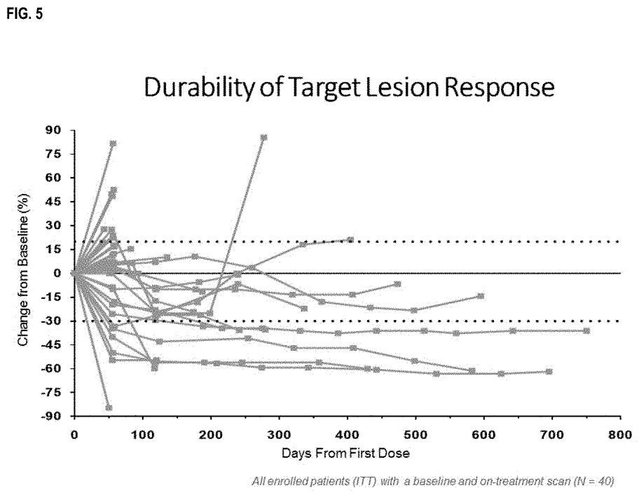

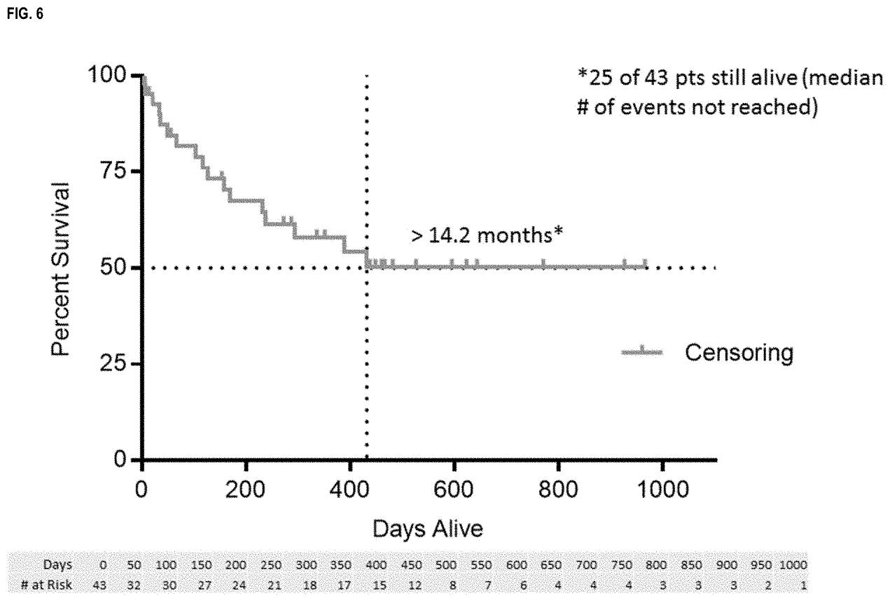

FIG. 6 shows the overall survival (OS) curve for the ITT population with a not yet reached median of >14.4 months. This population of patients only had a median of one prior course of treatment, which was chemotherapy. As such, the ITT population was CPI naïve, and when treated with nivolumab alone, the overall median survival was 12.2 months.

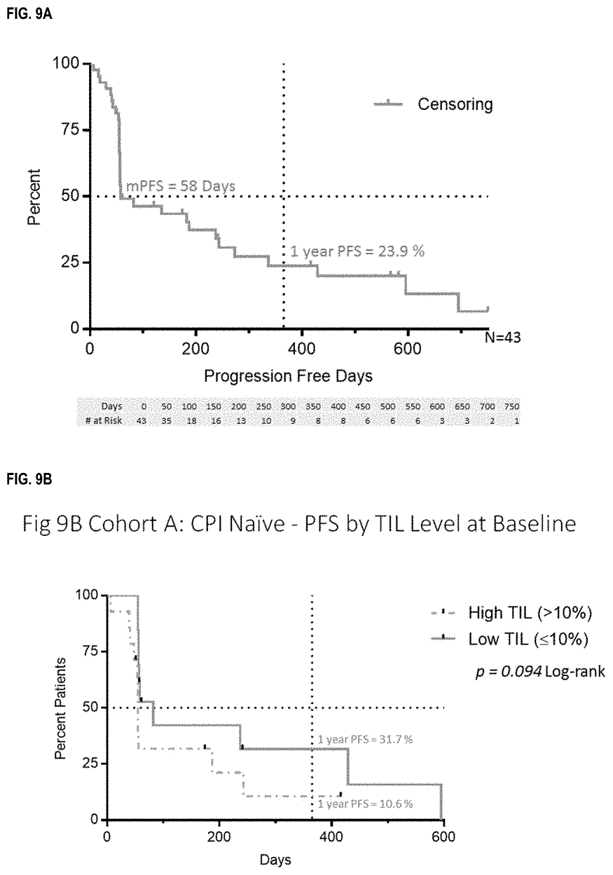

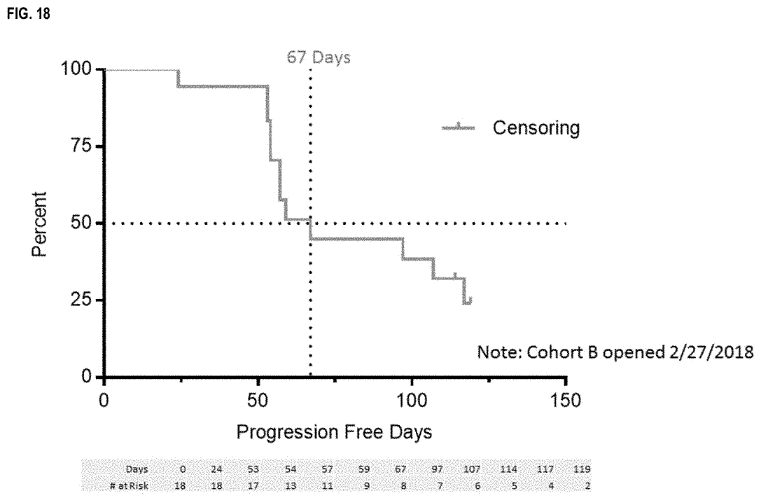

FIG. 9A and FIG. 9B are graphs showing progression free survival (PFS) in the CPI naïve ITT population (FIG. 9A), and the progression free survival by TIL level at baseline (FIG. 9B) in the CPI naïve ITT population. In FIG. 9A, the median PFS (mPFS) was 58 days and the 1 year PFS was 23.9%. In FIG. 9B, the 1 year PFS for low TILs was 31.7%, and the 1 year PFS for high TILs was 10.6%. Similarly, FIG. 18 is a plot showing PFS in the ITT patient population of patients previously received CPI therapy with disease progression after 6 months or longer where the mPFS was 67 days.

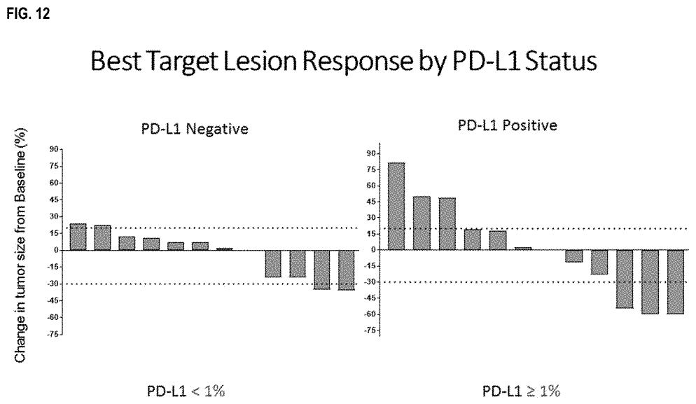

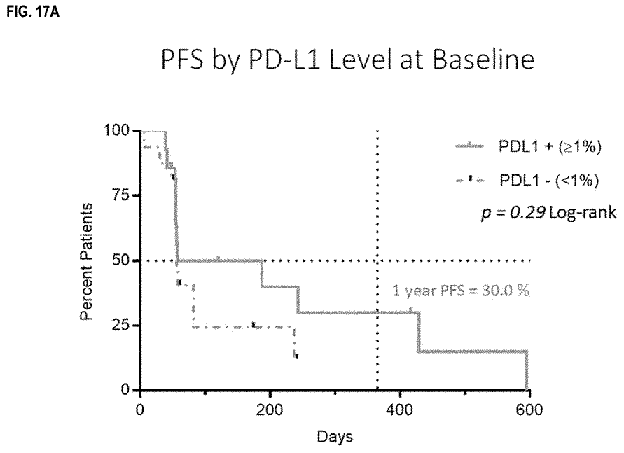

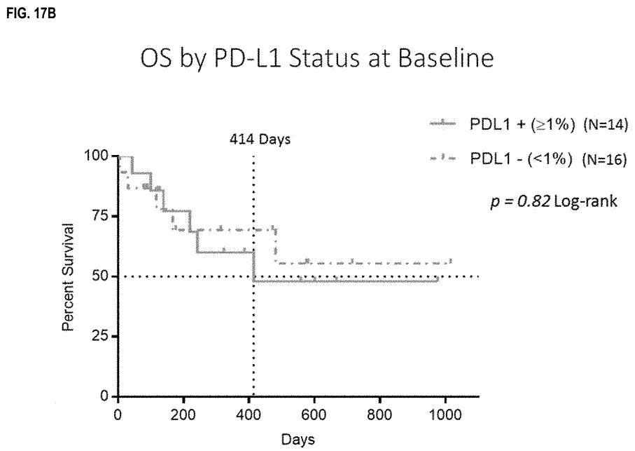

FIG. 17A and FIG. 17B are graphs showing the percentage of CPI naïve patients who experienced progression free survival (PFS) (FIG. 17A), or overall survival (OS) (FIG. 17B), by PD-L1 level at baseline. The 1 year PFS was 30% for the PD-L1+ (≥1%) patients. In terms of ORR, it is surprising that there is no difference in target lesion response by RESIST 1.1 based on PD-L1 status at baseline (FIG. 12)

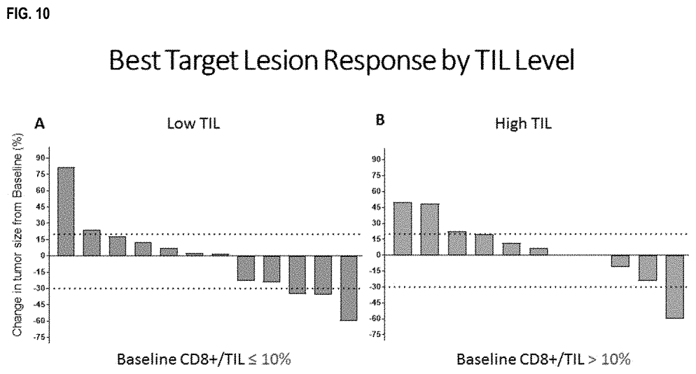

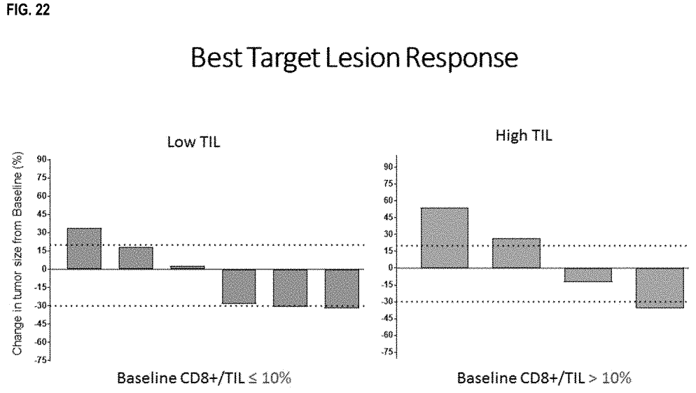

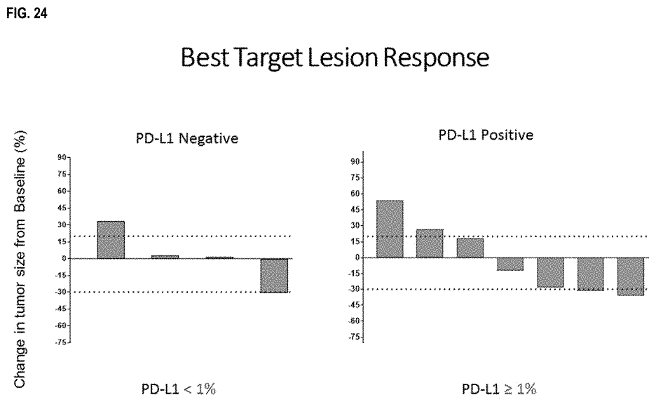

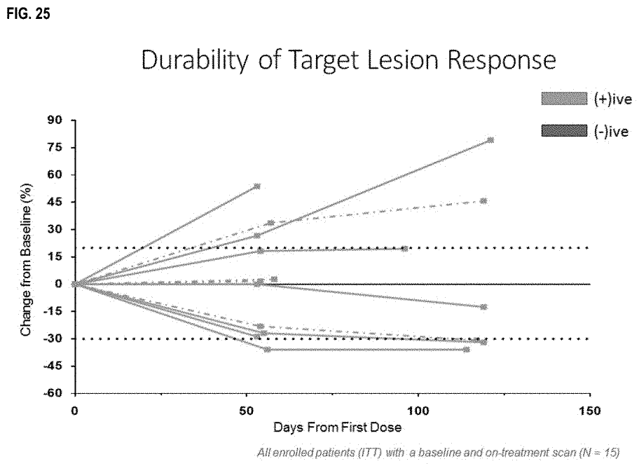

FIGS. 12-13 show the best target lesion response, and durability of target lesion response based on PD-L1 status in the per protocol population that was CPI naïve, or in the CPI progressor population (see FIG. 24, FIG. 25). Similarly, FIGS. 10A, 10B, and FIG. 11 show the best target lesion response and durability based on Tumor infiltrating lymphocytes (TIL) status for the per protocol population, which was CPI naïve. FIG. 22 shows the best target lesion response based on TIL status in the checkpoint inhibitor (CPI) progressor population. FIG. 23 shows the durability of target lesion response based on TIL level in the CPI progressor population.

The data disclosed herein shows that for cohort A, 14 patients (32.6%) were TIL high, 13 (30.2%) were TIL low, and 16 (37.2%) were TIL unknown. In cohort A, ORR, disease control rate (DCR), median progression-free survival (PFS), and 1 year PFS were 18.6%, 48.8%, 1.9 months and 23.9%, respectively, with median follow up of 432 days. In cohort B, where patients received both HS-110 and nivolumab, ORR, DCR, and PFS were 22%, 50% and 2.2 months, respectively, with median follow up of 43 days. The median overall survival (mOS) was not reached in either cohort. In cohort A, TIL low at baseline was associated with increased mOS compared to TIL high (not reached vs 13.8 months, hazard ratio [HR] 0.23, 95% CI 0.068-0.81, p=0.04). There were no differences in mOS according to PD-L1 status in cohort A (p=0.82). A total of 57 (93%) patients from both cohorts A and B experienced at least one adverse event (AE), of which 39 (64%) were grade 1 or 2. The most common AEs were fatigue (31%), cough and diarrhea (19.7% each). There were 2 grade 5 AEs (3.3%) caused by pulmonary embolism and tumor progression, neither considered to be treatment related

The results disclosed herein show that Nivolumab (anti-PD-1) is not efficacious alone in PD-L1^{negative} or PD-L1^{low} patients. However, combination therapy (HS-110+nivolumab) is equally effective regardless of PD-L1 status. Thus, this the combination expands nivolumab efficacy to the PD-L1_{neg} or PD-L1_{low} cancer patients, as well as TIL low patients at baseline.

In summary, the combination of viagenpumatucel-L (HS-110) and nivolumab (anti-PD-1) was a safe and effective treatment. Adaptive immune responses by ELISPOT correlated with clinical benefit in patients completing HS-110 treatment and with improved overall survival in the Intent-To-Treat population. Completion of study treatment with viagenpumatucel-L significantly improves overall survival when compared with non-completers (p=0.04). Similarly, low Tumor infiltrating lymphocyte (TIL) patients are not very responsive to nivolumab. However, combination therapy (HS-110+nivolumab is equally effective regardless of TIL status (e.g., similar effect in TIL_{low} as well as TIL_{high}, thus, the combination expands nivolumab efficacy to the TIL_{low} cancer patients to the point of a significant OS benefit over TIL_{high} patients. Moreover, combination treatment with viagenpumatucel-L and nivolumab resulted in dramatic infiltration of CD8⁺ T-cells into tumor tissue at week 10, and is associated with clinical responses of tumor reduction (Partial Response per RECIST 1.1).

Accordingly, without limitation, Examples 1 and 2 show the clinical success of viagenpumatucel-L (HS-110) plus nivolumab in patients with advanced non-small cell lung cancer (NSCLC). Patients with previously treated NSCLC received 1×10⁷ HS-110 cells weekly for the first 18 weeks and nivolumab 3 mg/kg or 240 mg every 2 weeks until intolerable toxicity or tumor progression. Tissue was tested at baseline for PD-L1 expression (≥1% or <1%) and tumor infiltrating lymphocytes (TILs). TIL high was defined by more than 10% CD8+ lymphocytes in the tumor stroma. Patients in cohort A had never received, and patients in cohort B had received, prior ICBs. The primary objectives were safety and objective response rates (ORR). There were 43 patients enrolled into cohort A (40 AD and 3 squamous cell carcinoma [SCC]) and 18 patients in cohort B (15 AD and 3 SCC). In cohort A, 14 patients (32.6%) were TIL high, 13 (30.2%) TIL low and 16 (37.2%) TIL unknown. ORR, disease control rate (DCR), median progression-free survival (PFS) and 1 year PFS were 18.6%, 48.8%, 1.9 months and 23.9% respectively in cohort A, with median follow up of 432 days. ORR, DCR, and PFS were 22%, 50% and 2.2 months respectively in cohort B, with median follow up of 43 days. The median overall survival (mOS) was not reached in either cohort. In cohort A, TIL low at baseline was associated with increased mOS compared to TIL high (not reached vs 13.8 months, hazard ratio [HR] 0.23, 95% CI 0.068-0.81, p=0.04). There were no differences in mOS according to PD-L1 status in cohort A (p=0.82). 57 (93%) patients experienced at least one adverse event (AE), of which 39 (64%) were grade 1 or 2. The most common AEs were fatigue (31%), cough and diarrhea (19.7% each). There were 2 grade 5 AEs (3.3%) caused by pulmonary embolism and tumor progression, neither considered to be treatment related.

SEQUENCES

Nucleotide sequence of full length human gp-96 (Genbank Accession No. X15187):
atgagggccctgtgggtgctgggcctctgctgcgtcctgctgaccttcgggtcggtcagagctgacgatgaagttgatgtggatggtacagtagaaga
ggatctgggtaaaagtagagaaggatcaaggacggatgatgaagtagtacagagagaggaagaagctattcagttggatggattaaatgcatcacaaa
taagagaacttagagagaagtcggaaaagtttgccttccaagccgaagttaacagaatgatgaaacttatcatcaattcattgtataaaaataaagag
attttcctgagagaactgatttcaaatgcttctgatgctttagataagataaggctaatatcactgactgatgaaaatgctctttctggaaatgagga
actaacagtcaaaattaagtgtgataaggagaagaacctgctgcatgtcacagacaccggtgtaggaatgaccagagaagagttggttaaaaaccttg
gtaccatagccaaatctgggacaagcgagtttttaaacaaaatgactgaagcacaggaagatggccagtcaacttctgaattgattggccagtttggt
gtcggtttctattccgccttccttgtagcagataaggttattgtcacttcaaaacacaacaacgatacccagcacatctgggagtctgactccaatga
attttctgtaattgctgacccaagaggaaacactctaggacggggaacgacaattacccttgtcttaaaagaagaagcatctgattaccttgaattgg
atacaattaaaaatctcgtcaaaaaatattcacagttcataaactttcctatttatgtatggagcagcaagactgaaactgttgaggagcccatggag
gaagaagaagcagccaaagaagagaaagaagaatctgatgatgaagctgcagtagaggaagaagaagaagaaaagaaaccaaagactaaaaaagttga
aaaaactgtctgggactgggaacttatgaatgatatcaaaccaatatggcagagaccatcaaaagaagtagaagaagatgaatacaaagctttctaca
aatcattttcaaaggaaagtgatgaccccatggcttatattcactttactgctgaaggggaagttaccttcaaatcaattttatttgtacccacatct
gctccacgtggtctgtttgacgaatatggatctaaaaagagcgattacattaagctctatgtgcgccgtgtattcatcacagacgacttccatgatat
gatgcctaaatacctcaattttgtcaagggtgtggtggactcagatgatctccccttgaatgtttcccgcgagactcttcagcaacataaactgctta
aggtgattaggaagaagcttgttcgtaaaacgctggacatgatcaagaagattgctgatgataaatacaatgatactttttggaaagaatttggtacc
aacatcaagcttggtgtgattgaagaccactcgaatcgaacacgtcttgctaaacttcttaggttccagtcttctcatcatccaactgacattactag
cctagaccagtatgtggaaagaatgaaggaaaaacaagacaaaatctacttcatggctgggtccagcagaaaagaggctgaatcttctccatttgttg
agcgacttctgaaaaagggctatgaagttatttacctcacagaacctgtggatgaatactgtattcaggcccttcccgaatttgatgggaagaggttc
cagaatgttgccaaggaaggagtgaagttcgatgaaagtgagaaaactaaggagagtcgtgaagcagttgagaaagaatttgagcctctgctgaattg
gatgaaagataaagcccttaaggacaagattgaaaaggctgtggtgtctcagcgcctgacagaatctccgtgtgctttggtggccagccagtacggat
ggtctggcaacatggagagaatcatgaaagcacaagcgtaccaaacgggcaaggacatctctacaaattactatgcgagtcagaagaaaacatttgaa
attaatcccagacacccgctgatcagagacatgcttcgacgaattaaggaagatgaagatgataaaacagttttggatcttgctgtggttttgtttga
aacagcaacgcttcggtcagggtatcttttaccagacactaaagcatatggagatagaatagaaagaatgcttcgcctcagtttgaacattgaccctg
atgcaaaggtggaagaagagcccgaagaagaacctgaagagacagcagaagacacaacagaagacacagagcaagacgaagatgaagaaatggatgtg
ggaacagatgaagaagaagaaacagcaaaggaatctacagctgaaaaagatgaattgtaa (SEQ ID NO: 1).
Amino acid sequence of the human gp96 gene of Genbank Accession No. CAA33261:
MRALWVLGLCCVLLTFGSVRADDEVDVDGTVEEDLGKSREGSRTDDEVVQREEEAIQLDGLNASQIRELREKSEKFA
FQAEVNRMMKLIINSLYKNKEIFLRELISNASDALDKIRLISLTDENALSGNEELTVKIKCDKEKNLLHVTDTGVGMTREE
LVKNLGTIAKSGTSEFLNKMTEAQEDGQSTSELIGQFGVGFYSAFLVADKVIVTSKHNNDTQHIWESDSNEFSVIADPR
GNTLGRGTTITLVLKEEASDYLELDTIKNLVKKYSQFINFPIYVWSSKTETVEEPMEEEEAAKEEKEESDDEAAVEEEE
EEKKPKTKKVEKTVWDWELMNDIKPIWQRPSKEVEEDEYKAFYKSFSKESDDPMAYIHFTAEGEVTFKSILFVPTSAP
RGLFDEYGSKKSDYIKLYVRRVFITDDFHDMMPKYLNFVKGVVDSDDLPLNVSRETLQQHKLLKVIRKKLVRKTLDMIK
KIADDKYNDTFWKEFGTNIKLGVIEDHSNRTRLAKLLRFQSSHHPTDITSLDQYVERMKEKQDKIYFMAGSSRKEAES
SPFVERLLKKGYEVIYLTEPVDEYCIQALPEFDGKRFQNVAKEGVKFDESEKTKESREAVEKEFEPLLNWMKDKALKD
KIEKAVVSQRLTESPCALVASQYGWSGNMERIMKAQAYQTGKDISTNYYASQKKTFEINPRHPLIRDMLRRIKEDEDD
KTVLDLAVVLFETATLRSGYLLPDTKAYGDRIERMLRLSLNIDPDAKVEEEPEEEPEETAEDTTEDTEQDEDEEMDVG
TDEEEETAKESTAEKDEL (SEQ ID NO: 2).
Retention Sequence of Gp96: KDEL (SEQ ID NO: 3).
Amino acid sequence of the human gp96 gene with the KDEL sequence deleted:
MRALWVLGLCCVLLTFGSVRADDEVDVDGTVEEDLGKSREGSRTDDEVVQREEEAIQLDGLNASQIRELREKSEKFA
FQAEVNRMMKLIINSLYKNKEIFLRELISNASDALDKIRLISLTDENALSGNEELTVKIKCDKEKNLLHVTDTGVGMTREE
LVKNLGTIAKSGTSEFLNKMTEAQEDGQSTSELIGQFGVGFYSAFLVADKVIVTSKHNNDTQHIWESDSNEFSVIADPR
GNTLGRGTTITLVLKEEASDYLELDTIKNLVKKYSQFINFPIYVWSSKTETVEEPMEEEEAAKEEKEESDDEAAVEEEE
EEKKPKTKKVEKTVWDWELMNDIKPIWQRPSKEVEEDEYKAFYKSFSKESDDPMAYIHFTAEGEVTFKSILFVPTSAP
RGLFDEYGSKKSDYIKLYVRRVFITDDFHDMMPKYLNFVKGVVDSDDLPLNVSRETLQQHKLLKVIRKKLVRKTLDMIK
KIADDKYNDTFWKEFGTNIKLGVIEDHSNRTRLAKLLRFQSSHHPTDITSLDQYVERMKEKQDKIYFMAGSSRKEAES
SPFVERLLKKGYEVIYLTEPVDEYCIQALPEFDGKRFQNVAKEGVKFDESEKTKESREAVEKEFEPLLNWMKDKALKD
KIEKAVVSQRLTESPCALVASQYGWSGNMERIMKAQAYQTGKDISTNYYASQKKTFEINPRHPLIRDMLRRIKEDEDD
KTVLDLAVVLFETATLRSGYLLPDTKAYGDRIERMLRLSLNIDPDAKVEEEPEEEPEETAEDTTEDTEQDEDEEMDVG
TDEEEETAKESTAE (SEQ ID NO: 4).
Amino acid sequence of the human sequence of the Fc domain absent the hinge region:
APEFLGGPSVFLFPPKPKDTLMISRTPEVTCVVVDVSQEDPEVQFNWYVDGVEVHNAKTKPREEQFNSTYRVVSVLT
VLHQDWLSGKEYKCKVSSKGLPSSIEKTISNATGQPREPQVYTLPPSQEEMTKNQVSLTCLVKGFYPSDIAVEWESN
GQPENNYKTTPPVLDSDGSFFLYSRLTVDKSSWQEGNVFSCSVMHEALHNHYTQKSLSLSLGK (SEQ ID NO: 5).

### Other Embodiments

It is to be understood that while the disclosure has been described in conjunction with the detailed description thereof, the foregoing description is intended to illustrate and not limit the scope of the disclosure, which is defined by the scope of the appended claims. Other aspects, advantages, and modifications are within the scope of the following claims.

### INCORPORATION BY REFERENCE

All patents and publications referenced herein are hereby incorporated by reference in their entireties. The publications discussed herein are provided solely for their disclosure prior to the filing date of the present application. Nothing herein is to be construed as an admission that the present invention is not entitled to antedate such publication by virtue of prior invention. As used herein, all headings are simply for organization and are not intended to limit the disclosure in any way.

What is claimed is:

1. A method of treating lung cancer, comprising administering (a) a cell harboring an expression vector comprising a nucleotide sequence that encodes a secretable vaccine protein and (b) an immune checkpoint inhibitor to a subject in need thereof.

2. The method of claim 1, wherein the immune checkpoint inhibitor inhibits an immune checkpoint gene.

3. The method of claim 1 or 2, wherein the immune checkpoint inhibitor comprises an antibody or antigen binding fragment thereof.

4. The method of claim 2, wherein the immune checkpoint gene is selected from Programmed cell death protein 1 (PD-1), Programmed death-ligand 1 (PD-L1), Programmed death-ligand 1 (PD-L2), Tumor necrosis factor receptor superfamily, member 4 (TNFRSF4), tumor necrosis factor receptor superfamily member 25 (TNFRSF25), Death receptor 3 (DR3), Tumor necrosis factor receptor superfamily member 9 (TNFRSF9), Glucocorticoid-induced TNFR-related protein (GITR), Cytotoxic T-lymphocyte-associated protein 4 (CTLA-4) and Lymphocyte-activation gene 3 (LAG-3).

5. The method of claim 2, wherein the immune checkpoint gene is PD-1 or PD-L1.

6. The method of any one of claims 1-5, wherein the immune check point inhibitor is an anti-PD-1 or anti-PD-L1 antibody or antigen binding fragment thereof.

7. The method of claim 6, wherein the anti-PD-1 antibody or antigen binding fragment thereof is selected from nivolumab, pembrolizumab, pidilizumab, BMS-936559, atezolizumab, avelumab, and the PD-L1 antibody or antigen binding fragment thereof is durvalumab.

8. The method of claim 6, wherein the anti-PD-1 antibody or antigen binding fragment thereof is nivolumab.

9. The method of claim 1, wherein the lung cancer is a small cell lung cancer.

10. The method of claim 1 or 9, wherein the lung cancer is a non-small cell lung cancer.

11. The method of any one of claim 1, 9 or 10, wherein the non-small cell lung cancer is adenocarcinoma

12. The method of any one of claim 1, 9 or 10, wherein the non-small cell lung cancer is squamous cell carcinoma or large cell lung cancer.

13. The method of claim 1, wherein the method reduces lung cancer recurrence.

14. The method of any one of the preceding claims, wherein the method increases the activation or proliferation of tumor antigen specific T cells in the subject.

15. The method of any one of the preceding claims, wherein the method increases the activation or the number of CD8+ T cells in the subject.

16. The method of claim 14, wherein the method increases the activation or the number of IFN-γ secreting CD8+ T cells in the subject.

17. The method of any one of the preceding claims, wherein the subject is treated with a low dose amount of the cell.

18. The method of any one of the preceding claims, wherein the subject is administered with about 100,000 cells, about 150,000 cells, about 200,000 cells, about 250,000 cells, about 300,000 cells, about 350,000 cells, about 400,000 cells, about 450,000 cells, about 500,000 cells, about 550,000 cells, about 600,000 cells, about 650,000 cells, about 700,000 cells, about 750,000 cells, about 800,000 cells, about 850,000 cells, about 900,000 cells, about 950,000 cells, or about 1,000,000 cells, or about 1,500,000 cells, or about 2,000,000 cells, or about 2,500,000 cells, or about 3,000,000 cells, or about 3,500,000 cells, or about 4,000,000 cells, or about 4,500,000 cells, or about 5,000,000 cells, or about 5,500,000 cells, or about 6,000,000 cells, or about 6,500,000 cells, or about 7,000,000 cells, or about 7,500,000 cells, or about 8,000,000 cells, or about 8,500,000 cells, or about 9,000,000 cells, or about 9,500,000 cells, or about 10,000,000 cells.

19. The method of any one of the preceding claims, wherein the subject exhibits a robust increase in immune response following administration.

20. The method of claim 18, wherein the robust increase in immune response is defined as an increase of at least 2 fold above the baseline in the activation or proliferation of CD8+ T cells.

21. The method of claim 18 or 19, wherein the CD8+ T cells secrete IFN-γ.

22. The method of any one of claims 19-21, wherein the method is more effective in reducing lung cancer recurrence in the subject compared to a subject who does not exhibit a robust increase in immune response.

23. The method of any one of the preceding claims, wherein the subject exhibits a low number of tumor infiltrating lymphocytes (TILs) prior to administration.

24. The method of claim 23, wherein the method is more effective in reducing cancer recurrence in the subject as compared to treatment with the immune checkpoint inhibitor alone.

25. The method of any one of the preceding claims, wherein the vector is a mammalian expression vector.

26. The method of any one of the preceding claims, wherein the vaccine protein is a secretable gp96-Ig fusion protein which optionally lacks the gp96 KDEL (SEQ ID NO:3) sequence.

27. The method of claim 26, wherein the Ig tag in the gp96-Ig fusion protein comprises the Fc region of human IgG1, IgG2, IgG3, IgG4, IgM, IgA, or IgE.

28. The method of any one of the preceding claims, wherein the expression vector comprises DNA.

29. The method of any one of the preceding claims, wherein the expression vector comprises RNA.

30. The method of any one of the preceding claims, wherein the cell is an irradiated or live and attenuated human tumor cell.

31. The method of claim 30, wherein the human tumor cell is a cell from an established NSCLC, bladder cancer, melanoma, ovarian cancer, renal cell carcinoma, prostate carcinoma, sarcoma, breast carcinoma, squamous cell carcinoma, head and neck carcinoma, hepatocellular carcinoma, pancreatic carcinoma, or colon carcinoma cell line.

32. The method of claim 30 or 31, wherein the human tumor cell line is a NSCLC cell line.

33. The method of any one of the preceding claims, wherein prior to the administering of (a) the cell harboring the expression vector comprising the nucleotide sequence that encodes the secretable vaccine protein, and prior to the administering of (b) the immune checkpoint inhibitor, the subject has experienced disease progression after receiving a therapy.

34. The method of claim 33, wherein the therapy is an immune checkpoint inhibitor therapy.

35. The method of claim 33 or 34, wherein the therapy comprises chemotherapy.

36. The method of any one of claims 33-35, wherein the subject is a poor responder to the immune checkpoint inhibitor therapy.

37. The method of any one of claims 33-36, wherein the subject has failed the immune checkpoint inhibitor therapy.

38. The method of any one of claims 33-37, wherein the disease in the subject has progressed even when administered the immune checkpoint inhibitor therapy.

39. A method of treating a patient with NSCLC comprising:
a) administering to said patient a weekly dose of HS-110 for at least 6 weeks; and
b) administering to said patient a biweekly dose of an anti-PD-1 antibody for at least 6 weeks.

40. A method of treating a patient with NSCLC with PD-L1^{negative} or PD-L1^{low} status comprising:
a) administering to said patient a weekly dose of HS-110 for at least 16 weeks; and
b) administering to said patient a biweekly dose of an anti-PD-1 antibody for at least 16 weeks.

41. A method of treating a patient with NSCLC with PD-L1^{negative} or PD-L1^{low} status comprising:
a) administering to said patient a weekly dose of HS-110 for at least 6 weeks; and
b) administering to said patient a biweekly dose of an anti-PD-1 antibody for at least 6 weeks.

42. A method of increasing the efficacy of anti-PD-1 therapy in a patient with NSCLC who is PD-L1^{negative} or PD-L1^{low} status comprising:
a) administering to said patient a weekly dose of HS-110 for at least 16 weeks; and
b) administering to said patient a biweekly dose of an anti-PD-1 antibody for at least 16 weeks.

43. A method of increasing the efficacy of anti-PD-1 therapy in a patient with NSCLC who is PD-L1^{negative} or PD-L1^{low} status comprising:
a) administering to said patient a weekly dose of HS-110 for at least 6 weeks; and
b) administering to said patient a biweekly dose of an anti-PD-1 antibody for at least 6 weeks.

44. A method of increasing the efficacy of anti-PD-1 therapy in a patient with NSCLC with low tumor infiltrating lymphocytes (TILs) status (TIL^{low}) comprising:
a) administering to said patient a weekly dose of HS-110 for at least 16 weeks; and
b) administering to said patient a biweekly dose of an anti-PD-1 antibody for at least 16 weeks.

45. A method of increasing the efficacy of anti-PD-1 therapy in a patient with NSCLC with low tumor infiltrating lymphocytes (TILs) status (TIL^{low}) comprising:
a) administering to said patient a weekly dose of HS-110 for at least 6 weeks; and
b) administering to said patient a biweekly dose of an anti-PD-1 antibody for at least 6 weeks.

46. A method according to any of claims 39-45 wherein said dose of HS-110 is about 1×10⁷ cells.

47. A method according to any of claims 39-46 wherein said dose of said anti-PD-1 antibody is 240 mg.

48. A method according to any of claims 39-47 wherein said anti-PD-1 antibody is selected from nivolumab and pembrolizumab.

49. A method of any one of the preceding claims, wherein the patient has experienced disease progression after receiving a therapy.

50. The method of claim 49, wherein the therapy is an immune checkpoint inhibitor therapy.

51. The method of claim 49 or 50, wherein the therapy comprises chemotherapy.

52. The method of any one of claims 49-51, wherein the patient is a poor responder to the immune checkpoint inhibitor therapy.

53. The method of any one of claims 49-52, wherein the patient has failed the immune checkpoint inhibitor therapy.

54. The method of any one of claims 49-53, wherein the disease in the patient has progressed even when administered the immune checkpoint inhibitor therapy.

55. The method of any one of claims 39-54, wherein the method reduces lung cancer recurrence.

56. The method of any one of claims 39-55, wherein the method increases the activation or proliferation of tumor antigen specific T cells in the subject.

57. The method of any one of claims 39-56, wherein the method increases the activation or the number of CD8+ T cells in the subject.

58. The method of claim 57, wherein the method increases the activation or the number of IFN-γ secreting CD8+ T cells in the subject.

59. The method of any one of claims 39-58, wherein the subject exhibits a robust increase in immune response following administration.

60. The method of claim 59, wherein the robust increase in immune response is defined as an increase of at least 2 fold above the baseline in the activation or proliferation of CD8+ T cells.

61. The method of claim 60, wherein the CD8+ T cells secrete IFN-γ.

62. The method of any one of claims 39-61, wherein the method is more effective in reducing lung cancer recurrence in the subject compared to a subject who does not exhibit a robust increase in immune response.

63. The method of any one of claims 39-62, wherein the subject exhibits a low number of tumor infiltrating lymphocytes (TILs) prior to administration.

64. The method of any one of claims 39-63, wherein the method is more effective in reducing cancer recurrence in the subject as compared to treatment with the immune checkpoint inhibitor alone.
